SEQUENCE LISTING (13 sequences)

SEQ ID NO: 1 - DNA, 23 bp, Artificial Sequence
  synthetic construct
cccggatgatgtcacagctcttc

SEQ ID NO: 2 - DNA, 20 bp, Artificial Sequence
  synthetic construct
tggcaacgggcactgctacc

SEQ ID NO: 3 - DNA, 20 bp, Artificial Sequence
  synthetic construct
tccagatggggcagagcctg

SEQ ID NO: 4 - DNA, 20 bp, Artificial Sequence
  synthetic construct
cctccttcctcctgagactc

SEQ ID NO: 5 - DNA, 20 bp, Artificial Sequence
  synthetic construct
acgggaactggtcacatacc

SEQ ID NO: 6 - DNA, 20 bp, Artificial Sequence
  synthetic construct
acatcatccgggtgtagtag

SEQ ID NO: 7 - DNA, 24 bp, Artificial Sequence
  synthetic construct
ggacacggagatcttccctggcaa

SEQ ID NO: 8 - DNA, 4 bp, Artificial Sequence
  synthetic construct
ggaa

SEQ ID NO: 9 - DNA, 23 bp, Artificial Sequence
  synthetic construct
ggacacggagatcttccctgcaa

SEQ ID NO: 10 - DNA, 21 bp, Artificial Sequence
  synthetic construct
cccgtgatgtcacagctcttc

SEQ ID NO: 11 - DNA, 18 bp, Artificial Sequence
  synthetic construct
ctgatgtcacagctcttc

SEQ ID NO: 12 - DNA, 90 bp, Artificial Sequence
  synthetic construct
tctgcccctcggacacggagatcttccctggcaacgggcactgctaccgcctggtggtggagaaggcggcctggctgcaggcgcaggagc

SEQ ID NO: 13 - DNA, 70 bp, Artificial Sequence
  synthetic construct
tctgcccctcggaacgggcactgctaccgcctggtggtggagaaggcggcctggctgcaggcgcaggagc

(19) United States
(12) Patent Application Publication
(10) Pub. No.: US 2020/0390825 A1
(43) Pub. Date: Dec. 17, 2020

(54) PLURIPOTENT STEM CELL-DIRECTED MODEL OF AUTOSOMAL DOMINANT POLYCYSTIC KIDNEY DISEASE FOR DISEASE MECHANISM AND DRUG DISCOVERY

(71) Applicant: University of Southern California, Los Angeles, CA (US)

(72) Inventors: Andrew MCMAHON, Los Angeles, CA (US); Trinh TRAN, Los Angeles, CA (US); Cheng SONG, Los Angeles, CA (US)

(73) Assignee: University of Southern California, Los Angeles, CA (US)

(21) Appl. No.: 16/900,731

(22) Filed: Jun. 12, 2020

Related U.S. Application Data
(60) Provisional application No. 62/861,946, filed on Jun. 14, 2019.

(51) Int. Cl. A61K 35/545; C12N 5/071; C12N 5/10; A61K 35/22; G01N 33/68

(52) U.S. Cl. CPC A61K 35/545; C12N 5/0686; C12N 5/10; A61K 35/22; G01N 33/6803; C12N 2513/00; C12N 2501/115; C12N 2501/999; C12N 2506/03; C12N 2506/45; C12N 2501/727

(57) ABSTRACT

A new type of kidney miniature organoids based on human embryonic stem cells are prepared and tested as forming cysts in vitro or ex vivo. Assays are developed for screening useful candidate molecules towards inhibiting or treating polycystic kidney disease. This provides a new system for modeling polycystic kidney disease.

### CROSS-REFERENCE TO RELATED APPLICATIONS

This application includes a claim of priority under 35 U.S.C. § 119(e) to U.S. provisional patent application No. 62/861,946, filed Jun. 14, 2019, the entirety of which is hereby incorporated by reference.

### REFERENCE TO SEQUENCE LISTING

The Sequence Listing submitted Aug. 18, 2020, as a text file named “AmendedSequenceListing-065715-000100US00_ST25” created on Jul. 29, 2020 and having a size of 3,097 bytes, is hereby incorporated by reference, which replaces the sequence listing submitted on Jun. 12, 2020 and includes no new matter.

### FIELD OF INVENTION

This invention relates to methods of enhancing development of renal organoids, methods of using the same, and kits.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Kidney disease affects about 30 million Americans or 15 percent of U.S. adults. Autosomal dominant polycystic kidney disease (ADPKD) is a most common genetic renal disorder. Mutations in either of two genes, polycystin1 (PKD1) and polycystin2 (PKD2), account for the majority of ADPKD cases, in which tubular epithelia form fluid-filled cysts. Overgrowth of fluid-filled renal cysts is associated with the loss of renal function, which can result in end-stage renal failure leading to death. The disease is considered to be a systemic disorder, characterized by cyst formation in the ductal organs such as kidney, liver, and pancreas, as well as by gastrointestinal, cardiovascular, and musculoskeletal abnormalities, including colonic diverticulitis, berry aneurysms, hernias, and mitral valve prolapse.

The smallest functional unit of the kidney that helps remove blood waste from the body are nephrons. Nephrons are formed in the human kidney only during fetal life, before the stem cells that generate them are exhausted. A major barrier to understanding polycystic kidney disease (PKD) is the absence of human cellular models that accurately and efficiently recapitulate cystogenesis.

Therefore, there is a great need in the art for diagnostic and therapeutic tools to reduce the incidence and severity of this disease by faithful modeling the disease and identification of drugs with specificity to treat the kidney disease.

### SUMMARY OF THE INVENTION

The following embodiments and aspects thereof are described and illustrated in conjunction with compositions and methods which are meant to be exemplary and illustrative, not limiting in scope.

Methods of generating a nephronic lineage organoid are provided, which includes differentiating pluripotent stem cells (PSCs) in a process including:

culturing a quantity of the PSCs in the presence of a first small molecule, and culturing the quantity of PSCs in the presence of at least one growth factor, to generate nephron progenitor-like cells;

culturing the nephron progenitor-like cells in the presence of the at least one growth factor and a second small molecule, and generating a pellet of cells by placing the nephron progenitor-like cells into a microwell;

incubating the pellet of cells in the presence of the at least one growth factor; and

further culturing the pellet of cells in the absence of the at least one growth factor to generate a nephronic lineage organoid.

In some embodiments, the at least one first growth factor comprises activin, fibroblast growth factor (FGF) 9, or both; and at least one of the first small molecule and the second small molecule is CHIR99021.

In further embodiments, a chimeric nephronic lineage organoid is generated wherein the PSCs in the methods contain a quantity that have mutant polycystin-1 gene and/or mutant polycystin-2 gene and another quantity that have normal polycystin-1 gene and normal polycystin-2 gene.

In some embodiments, the chimeric nephronic lineage organoid is generated with at least a quantity of PSCs that express marker genes, including but are not limited to a podocyte marker, a proximal tube marker, a loop of Henle marker, and a distal tubule marker.

Nephronic lineage organoids are provided with the generation process, which at least 10%, 20%, 30%, 40%, 50%, 60%, or more in quantity develops cysts.

Various embodiments provide the nephronic lineage organoids are cultured in a viscous medium, such as one comprising an effective amount of a polymer (e.g., methylcellulose) for reducing vibration or artifacts that can be caused by a user's maneuvering in order to allow for multiday imaging and/or high resolution quantitative analysis.

Method of assessing one or more agents using the nephronic lineage organoids are also provided, which generally include contacting an agent of interest with the organoids and measuring the level of biomarkers in the organoids.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 is a schematic showing a process of generating kidney organoids on a well-plate from hESCs.

FIG. 2 is a schematic showing a process of seeding hESCs into a well-plate and forming pellets of hESCs.

FIG. 3 is a schematic showing an exemplary cocktail (reagents, doses, and timeline) for inducing hESCs into nephronic lineage cells.

FIG. 4 is a schematic showing a process of generating kidney miniature organoids embedded in a medium of methylcellulose from hESCs.

FIG. 5 is a diagram showing PKD2 mutation by a CRISPER-Cas9 system and inclusion of four reporter genes in ES cell line.

FIG. 6 is a line graph showing the progression of induced PKD2 mutation and four reporters in the embedded organoids.

FIG. 7 is a schematic diagram of directed differentiation to generate miniature kidney organoids.

FIG. 8 is a series of images (bright field overlaid with fluorescent) of miniature kidney organoids derived from MAFB-P2A-eGFP H9 hESC. Scale bars indicate 50 μm unless labeled differently.

FIG. 9 is a graph showing the average expression level (scoring from 1-5, indicated by intensity of circles) and percentage expressed (indicated by size of circles) of various markers in different cluster identities of the miniature organoids.

FIG. 10 is a diagram showing the cellular diversity in the miniature organoids, * selected for downstream nephrogenic analyses.

FIG. 11A is a graph showing the average expression level (indicated by intensity of circles) and percentage expressed (indicated by size of circles) of various markers in re-clustered nephrogenic clusters of 14,566 cells from the miniature organoids. FIG. 11B is a graph showing origins contributing to various nephrogenic identities in the re-clustered nephrogenic cells.

FIG. 12A is a chart showing nephrogenic cell types in cluster identification. FIG. 12B is a graph showing origins contributing to various nephrogenic identities.

FIG. 13A is a diagram of using CRISPR-Cas9 technology to generate PKD1−/− line on the background of H9 hESC cell line, in which relevant region's sequence of the wild type, the corresponding edited allele 1, and the corresponding edited allele 2 are shown as SEQ ID NOs:7, 8, and 9, respectively. FIG. 13B is a diagram of using CRISPR-Cas9 technology to generate PKD2−/− line on the background of H9 hESC cell line, in which relevant region's sequence of the wild type, the corresponding edited allele 1, and the corresponding edited allele 2 are shown as SEQ ID NOs:1, 10, and 11, respectively.

FIG. 14 is a graph showing PKD2 mRNA levels in mutation clone, fetal human kidney and in H9 wild type control from qPCR quantifications.

FIG. 15 is a diagram showing the procedure of embedding organoids in methylcellulose media and imaging them to track cyst development in mutated organoids.

FIG. 16 is a panel of representative microscopic images of the mutated and control organoids over time.

FIG. 17A is a graph showing the area/size of organoids of control and PKD1−/− organoids overtime. FIG. 17B is a graph showing the area/size of organoids of control and PKD2−/− organoids overtime.

FIG. 18A is a graph showing the percentage of cyst forming PKD1−/− organoids over time. FIG. 18B is a graph showing the percentage of cyst forming PKD2−/− organoids over time.

FIG. 19 is a schematic diagram describing the screening process to identify protein kinase inhibitors impeding cyst formation.

FIG. 20 is a schematic of cyst production, including a representative bright-field images of PKD2−/− organoid cyst cultured in Ad-RPMI medium for 4 weeks (scale bars, 1 mm).

FIG. 21A is a volcano plot showing, between PKD1 cyst and PKD1 organoid, the differentially expressed (DE) genes selected for PKD related genes, including YAP directly targeted genes are highlighted in bold. Negative log 2 fold change (PKD1c/PDK1o): PAK1, JARID2, P3H2, SLIT2, PRODH2, NME6, AGXT2, MT1X, SOCS2, MT1G/MT1H, FXN/SMAD4, AGT, HNF4A/WT1, MT1F/FGFR3, and SERTAD3. Positive log 2 fold change (PKD1c/PDK1o): CCN2, AMTL2, STAT1, CCN1, JAK1, C3, TNFRSF 12A, PROM1, SPP1, TUBB6, WWC1, EGFR, IGFBP6, HAVCR1, IGFBP7, COL12A1, GADD45B, CD44, SERPINE1, and S100A6.

FIG. 21B is a volcano plot showing, between PKD2 cyst and PKD2 organoid, the differentially expressed (DE) genes selected for PKD related genes, including YAP directly targeted genes are highlighted in bold. Negative log 2 fold change (PKD2c/PDK2o): HNF4A, PRODH2, AGT, WT1, AGXT2, PAK1, SLIT2, FGFR3, FXNNME6, MT1H, MT1G, MT1X, JARID2, SMAD4, SOCS2, SERTAD3, MT1F, and P3H2. Positive log 2 fold change (PKD2c/PDK2o): JAK1, CCN2, AMOTL2, CCN1, TUBB6, STAT1, S100A6, TNFRSF12A, COL12A, EGFR, GFBP6, PROM1, GADD45B, SERPINE1, CD44, SPP1, IGFBP7, WWC1, HAVCR1, and C3.

FIG. 21C is a graph showing overlap between cystic genes (PKD1 and PKD2) identified by differential gene expression analysis between PKD cyst and PKD organoid (FTPM >5, P<0.05).

FIGS. 21D and 21E are heatmaps/hierarchical clusters with expression levels of the 50 direct YAP/TAZ target genes that are commonly differentially expressed in both PKD1 and PKD2 cysts compared to PKD organoids.

FIG. 22 is a chart showing the fraction of cells displaying preferential nuclear YAP localization (lowest section), even distribution of YAP in nucleus and cytoplasm (middle section), or cytoplasmic YAP (top section). Data from approximately 200 cells from 10 random fields of view.

FIG. 23A is a schematic representation of the generation of a chimeric cyst using human ES cells. FIG. 23B is a series of microscopic images, where top three images bright field and fluorescence images showing the cyst formation on day 19 chimeric organoids but not EGFP-WT organoids (black arrowheads indicate cyst; scale bar, 200 μm); the middle three images are representative overlays (bring field plus fluorescence) illustrating cyst development from day 13 to 19 (scale bar, 50 μm); and the images labeled “20× Zoom” are higher magnification images of boxed region in the Day 19 image (scale bar, 50 μm). FIG. 23C is a cyst quantification of organoids 19 days after differentiation (n=3 separate experiments, more than 30 organoids, *** P=0.001, **** P<0.001). FIG. 23D is a bar graph showing YAP localization at 19 days of culture are mostly within cytoplasmic region (middle section) of wildtype (“GFP+”) and PKD2−/− (“GFP−”) cysts. FIG. 23E is a bar graph showing YAP localization in wildtype (“GFP+”) and PKD1−/− (“GFP−”) cysts of 1 mm diameter size, where nuclear localization of YAP1 was observed in PKD1−/− cells.

FIG. 24A is a schematic representation of the infection of a PKD2 cyst using adenovirus with mCherry reporter. FIG. 24B is a bar graph showing the percentage of cilia (ARL13B) colocalized with PKD2 signal. Data from approximately 100 cells from 10 random fields of view. FIG. 24C is a bar graph showing EdU incorporation rates of mCherry+ cells infected with either PKD2 overexpression or control virus as indicated. Data from 500 cells. FIG. 24D is a bar graph showing YAP nucleo/cytoplasmic localization of mCherry+ cells.

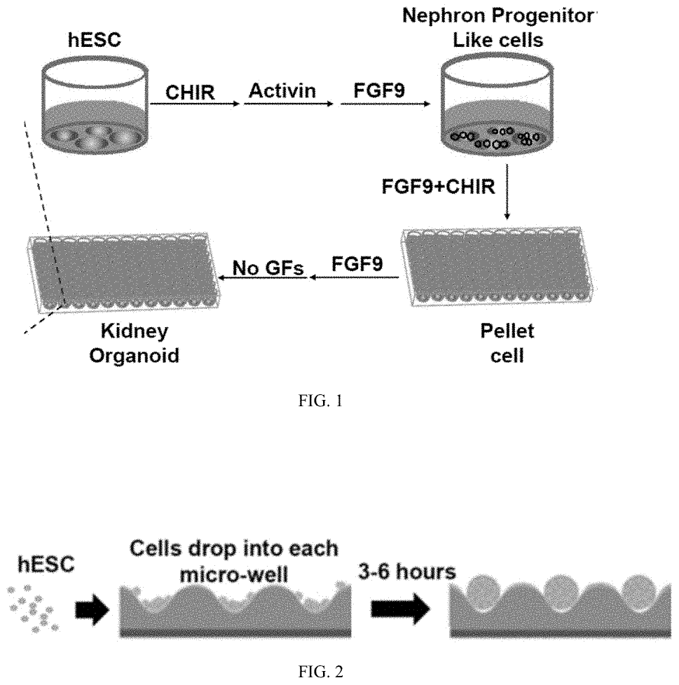

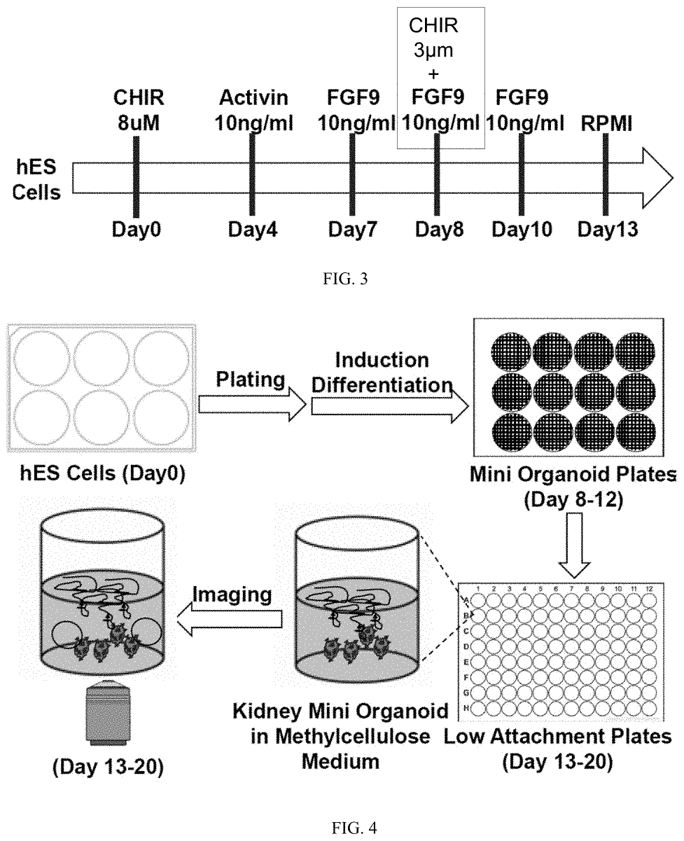

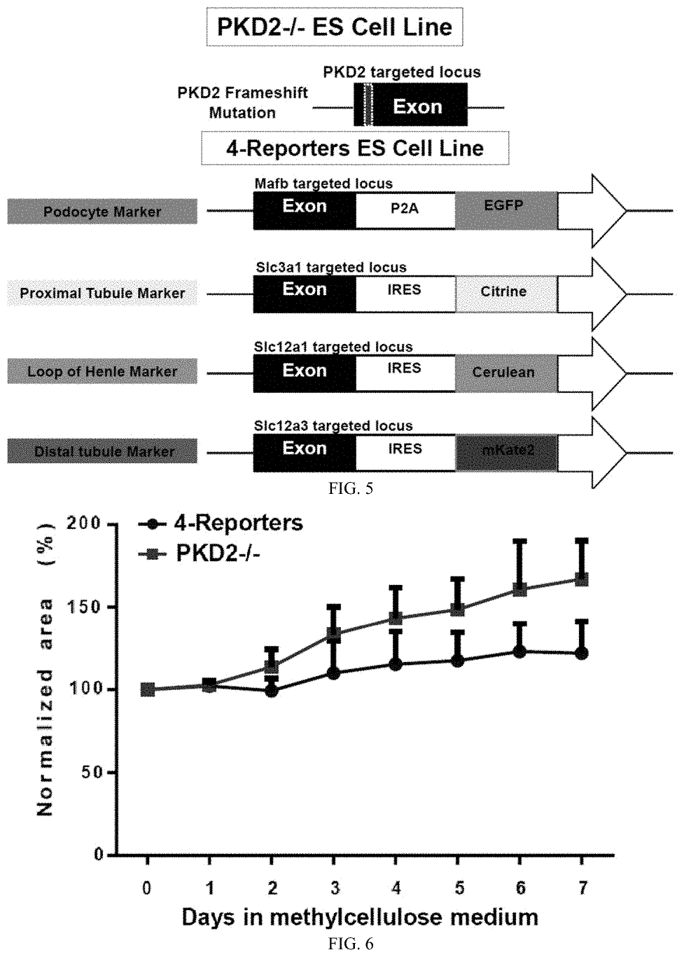

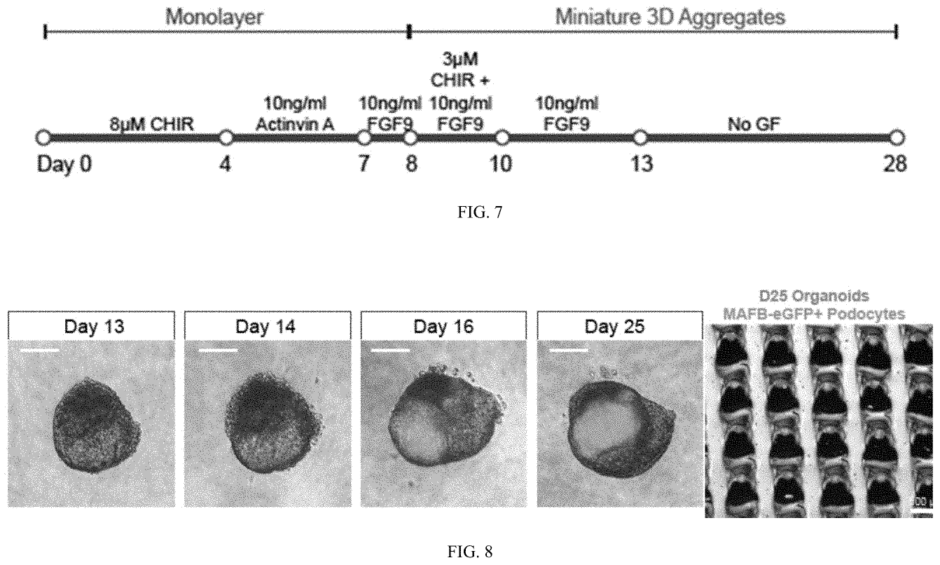

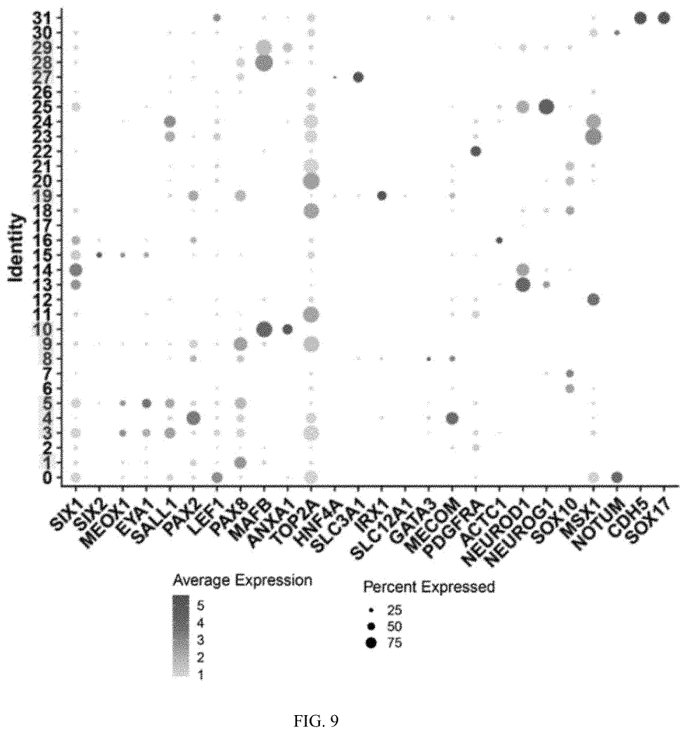

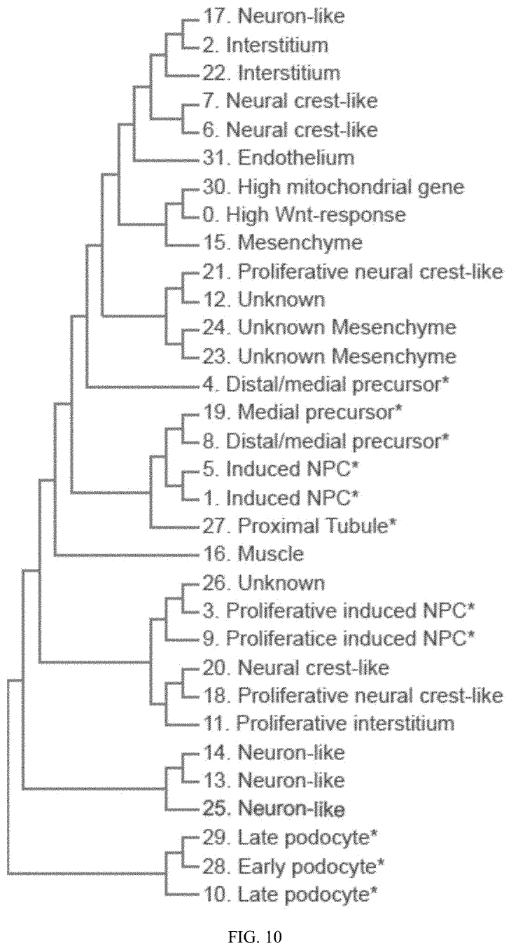

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The terms “nephrotic lineage organoid” or “renal organoid” can be used interchangeably and refer to a three-dimensional tissue culture created or synthesized by culturing one or several types of cells, e.g., human embryonic, pluripotent or multipotent stem cells on or embedded in a substrate, which have undergone a degree of differentiation. Nephrotic lineage organoids, or renal organoids, are formed into a three-dimensional sphere, spheroid, or other three dimensional shape, and have anatomical features that resemble mammalian kidneys, such as tubule structures as well as the same or similar, or partial functional features as the mammalian kidneys.

The term “nephron progenitor-like cell(s)” is generally used to describe cells with structural and/or functional similarity to nephron progenitors. In various embodiments, nephron progenitors can form an epithelial tubule (sometimes referred to as a renal vesicle) and give rise to proximal nephron, and therefore nephron progenitor-like cells may have the morphology, the expression profile, and/or the function of nephron progenitors. In some embodiments, nephron progenitor-like cells describe pluripotent stem cells after sequential cultivation with CHIR99021, activin, and FGF9, especially in a 2-D culture.

The term “growth factor” is used generally consistent with the meaning in the art. It describes a naturally occurring substance capable of stimulating cellular growth, proliferation, healing, and/or cellular differentiation. In various embodiments, a growth factor is a protein. In some embodiments, a growth factor includes a protein hormone.

The term “small molecule” is used generally consistent with the meaning in the art. In various embodiments, it refers to compounds manufactured through chemical synthesis; and thus they generally have well-defined chemical structures. In one embodiment, small molecules comprise CHIR99021, whose chemical name is 6-[[2-[[4-(2,4-dichlorophenyl)-5-(5-methyl-1H-imidazol-2-yl)-2-pyrimidinyl]amino]ethyl]amino]-3-pyridinecarbonitrile OR 3-Pyridinecarbonitrile, 6-[[2-[[4-(2,4-dichlorophenyl)-5-(4-methyl-1H-imidazol-2-yl)-2-pyrimidinyl]amino]ethyl]amino]-(9CI).

A “mutant” gene, or a mutated gene, is described herein as a gene whose sequence has been modified by transitions, transversions, deletions, insertions, or other modifications, which in most embodiments have apparent effect on expression or function of the gene product. In some embodiments, a normal gene has allelic variants that are not associated with disease and are considered to be a wild-type version of the gene, and hence a mutant gene is relative to a normal gene in terms of effecting a different or lack of function of the gene product, thereby being associated with a disease.

The term “PKD1 gene” refers to a genomic DNA sequence which maps to chromosomal position 16p13.3 and gives rise to a messenger RNA molecule encoding the PKD1 protein.

A “normal” PKD1 (or PKD2) gene is defined herein as a PKD1 (or PKD2) gene whose altered, defective, or non-functional expression leads to adult-onset polycystic kidney disease. A normal PKD1 (or PKD2) gene is not associated with disease and thus is considered to be a wild-type version of the gene. Included in this category are allelic variants in the PKD1 (or PKD2) gene, also denoted allelic polymorphisms, i.e. alternate versions of the PKD1 (or PKD2) gene, not associated with disease, that may be represented at any frequency in the population. Also included are alterations in DNA sequence, whether recombinant or naturally occurring, that have no apparent effect on expression or function of the PKD1 (or PKD2) gene product.

A “mutant” PKD1 (or PKD2) gene is used herein as a PKD1 (or PKD2) gene whose sequence has been modified by transitions, transversions, deletions, insertions, or other modifications relative to the normal PKD1 (or PKD2) gene, which modifications cause detectable changes in the expression or function of the PKD1 (or PKD2) gene product, including causing disease. The modifications may involve from one to as many as several thousand nucleotides, and result in one or more of a variety of changes in PKD1 (or PKD2) gene expression, such as, for example, decreased or increased rates of expression, or expression of a defective RNA transcript or protein product. Mutant PKD1 (or PKD2) genes encompass those genes whose presence in one or more copies in the genome of a human individual is associated with adult-onset polycystic kidney disease.

In various embodiments, we have generated PKD2 mutant human ESCs which form cystic kidney mini-organoid cultures of approximately 1000 cells in EZSPHERE plates, and employed a three-dimensional (3D) culture system to emulate cystic structures in vitro to analyze cyst initiation. This system was developed by culturing polycystin2 mutant and normal organoids modified to report on development of podocytes in cell culture medium with methylcellulose. In this system, the methylcellulose reduces organoid motility and maintains 3D structure. Together, the new mini organoid-based 3D culture system proves to be a new human model of PKD to assess the function of polycystins at an earliest disease stage.

In various embodiments, we have targeted huESC-09 human pluripotent stem cells to remove activity of both copies of the human polycystic kidney disease-2 (PKD2) gene. In some embodiments, thousands of in vitro kidney-like mini-organoids are produced, which undergo spontaneous cyst formation. In some embodiments, we have performed screens to identify genes that are specifically activated within cystic epithelial cells as well as signaling pathways that correlate with cytogenesis.

The system is configured for molecular, cellular and biochemical exploration of disease causing mechanisms. Screening assays based on the organoid system are conceived to test FDA-approved drugs for prevention, intervention, and/or treatment of polycystic kidney diseases. Application of the mini-organoid platform can be used for screening, optimized imaging for an extended period of time (e.g., coupled with a culture medium that comprises methylcellulose to increase viscosity and reduce sample vibration), genetic modification (gene targeted and reporters in cells), RNA screen for new genes, new chimeric cyst assay to analyze and visualize cyst generating process.

Methods of Generating

Methods of growing, generating, and/or preparing a nephronic lineage organoid are provided, which include differentiating a quantify of pluripotent stem cells (PSCs) in phases of a) cultivating (or culturing or incubating) the PSCs in a basal medium with the addition of a small molecule, b) cultivating the PSCs in a basal medium with the addition of at least one growth factor, c) cultivating the PSCs in a basal medium with a combination of the small molecule and the at least one growth factor, and d) cultivating the PSCs in a basal medium without the small molecule or the at least one growth factor.

Various embodiments of the methods of growing, generating, and/or preparing the nephronic lineage organoid includes differentiating the quantity of PSCs in phases of a sequence of a)-b)-c)-d), which is a) cultivating the PSCs in a basal medium with the small molecule for a first period of time, followed by b) cultivating the PSCs in a basal medium with the at least one growth factor for a second period of time, followed by c) cultivating the PSCs in a basal medium with a combination of the small molecule and the at least one growth factor for a third period of time, and subsequently d) cultivating the PSCs in a basal medium without the small molecule or the at least one growth factor for the fourth period of time. In some embodiments, step a) is cultivating in a basal medium with the small molecule, but absent from adding any growth factor on top of the basal medium and the small molecule. In some embodiments, step b) is cultivating in adding at least one growth factor on top of the basal medium, but no small molecule is added on top of the basal medium plus the at least one growth factor. In some embodiments, step d) is cultivating in only the basal medium.

In some embodiment, the sequence of phases has a repeated phase b), e.g., in a sequence of a)-b)-c)-b)-d). In some embodiments, phase b) has at least two sequentially added growth factors, i.e., b1) and b2), thereby the sequence being a)-b1)-b2)-c)-d); a)-b1)-b2)-c)-b1)-d); a)-b1)-b2)-c)-b2)-d); or a)-b1)-b2)-c)-b1)-b2)-d).

In some embodiments of the phases, any of the first, second, third, and fourth periods of time, or the period of time for the repeated phase b), is independently selected from about 1 day, 2 days, 3 days, 4 days, 5 days, 6 days, 7 days, 8 days, 9 days, 10 days, 11 days, 12 days, 13 days, 14 days, or in a range between any of the two mentioned lengths of time. In some embodiments, the first period of time for phase a) is about 4 days, or between about 3 days and 5 days. In some embodiments, the second period of time for phase b) is about 4 days, or between about 3 days and 5 days, which in the case of b1) and b2) totals about 4 days or between about 3 days and 5 days. In further embodiments the second period of time includes about 3 days for phase b1) followed by about 1 day for phase b2). In some embodiments, the third period of time for phase c) is about 2 days, or between about 1 day and 3 days. In some embodiments, the fourth period of time for phase d) is at least 2 days, or for as long as a user intends to grow the organoids. In further embodiments where phase b) is repeated after phase c), the period of time for the repeated phase b) is about 3 days, or between about 1 day and 5 days, before the initiation of phase d).

In some embodiment, the methods comprise first culturing the PSCs on a flat surface, and subsequently generating a pellet of the PSCs by plating the PSCs in a low-binding surface (e.g., EZSPHERE microwell plate), so as for the cells to ball up. In further embodiments, the methods further comprise embedding or suspending the pellet of cells in a matrix or viscous medium, such as methylcellulose network. In yet other embodiments, the basal medium of any one or more of the steps above is supplemented with methylcellulose to increase viscosity of the medium, thereby reducing vibration of the organoids caused by environment or maneuvering.

The step of generating a pellet of cells (or pelletizing) can be performed in between two phases in the sequence. In some embodiments, the step of pelletizing is performed after phase c), i.e., after the cultivation in the presence of both a growth factor and a small molecule. In some embodiments, the step of pelletizing is performed before phase c), i.e., the pellet of cells undergoes the cultivation phase in the presence of both a growth factor and a small molecule.

The step of pelletizing usually begins with seeding a density of about 600,000 cells, between about 500,000 cells and 700,000 cells, between about 400,000 cells and 800,000 cells, or between about 300,000 cells and 900,000 cells, in a well of about a surface area of 3.8 cm² (e.g., a well in a 12-well plate), thereby achieving about a few hundred pellets per well, wherein each pellet of about 1,000 cells, 1,100 cells, 1,200 cells, 1,300 cells, 1,400 cells, 1,500 cells, 1,600 cells, 1,700 cell, 1,800 cells, 1,900 cells, or 2,000 cells can grow into one organoid.

In some embodiments, the methods of generating a nephronic lineage organoid comprise:

culturing a quantity of pluripotent stem cells (PSCs) in the presence of a first small molecule, and culturing the quantity of PSCs in the presence of at least one growth factor, to generate nephron progenitor-like cells;

culturing the nephron progenitor-like cells in the presence of the at least one growth factor and a second small molecule, and generating a pellet of cells by placing the nephron progenitor-like cells into a microwell, wherein the order of these two steps can optionally be reversed;

incubating the pellet of cells in the presence of the at least one growth factor; and

further culturing the pellet of cells in the absence of the at least one growth factor to generate a nephronic lineage organoid.

In some embodiments, the methods of generating a nephronic lineage organoid comprise:

culturing a quantity of human PSCs in the presence of at least one first growth factor to generate nephron progenitor-like cells;

further culturing the nephron progenitor-like cells in the presence of at least one growth factor and at least one small molecule;

generating a pellet of cells by placing the nephron progenitor-like cells into a microwell;

incubating the pellet of cells with at least one growth factor; and

further culturing the pellet of cells in the absence of growth factor, thereby generating the nephronic lineage organoid.

In various embodiments, the small molecule comprises CHIR99021, which is a glycogen synthase kinase (GSK) 3 inhibitor. In some embodiments, the small molecule comprises Y27632, which is an inhibitor of Rho-associated kinase. CHIR99021, another GSK3 inhibitor, Y27632, or another Rho-associated kinase, can be used as the small molecule at a concentration of about 0.1-1 μM, 1-2 μM, 2-3 μM, 3-4 μM, 4-5 μM, 5-6 μM, 6-7 μM, 7-8 μM, 8-9 μM, 9-10 μM, 10-15 μM, 15-20 μM, 20-50 μM, 50-100 μM, or any range in between, in a basal medium for cultivating PSCs. In some embodiments, CHIR99021 or another GSK3 inhibitor is used in phase a) at a concentration of about 8 μM, or between about 7 μM and 9 μM. In some embodiments, CHIR99021 or another GSK3 inhibitor is used in phase c) at a small molecule concentration of about 3 μM, or between about 1 μM and 5 μM. In some embodiments, the small molecules do not comprise those in B-27; and the methods do not involve culturing the cells in the presence of B-27.

In various embodiments, the growth factors comprise activin, FGF9, or both. In some embodiments, activin is activin A. Each of the growth factor can be used at a concentration of about 0.1-1 ng/mL, 1-2 ng/mL, 2-3 ng/mL, 3-4 ng/mL, 4-5 ng/mL, 5-6 ng/mL, 6-7 ng/mL, 7-8 ng/mL, 8-9 ng/mL, 9-10 ng/mL, 10-11 ng/mL, 11-12 ng/mL, 12-13 ng/mL, 13-14 ng/mL, 14-15 ng/mL, 15-25 ng/mL, 25-50 ng/mL, 50-100 ng/mL, 100-1,000 ng/mL, or any range in between, in a basal medium. In some embodiments, activin and FGF9 are sequentially used in phase b), each at about 10 ng/mL, or between about 5-15 ng/mL. In some embodiments, FGF9 is used in phase c) in combination with the small molecule, and the FGF9 in phase c) is at about 10 ng/mL, or between about 5-15 ng/mL. In some embodiments, a repeated phase b) involves FGF9 at about 10 ng/mL, or between about 5-15 ng/mL. In some embodiments of the methods, the step of incubating the pellet of cells in the presence of the at least one growth factor comprises addition of FGF9 to the pellet of cells. In some embodiments, the growth factors do not comprise insulin, or the growth factors do not come from B-27.

Yet in some embodiments, the methods involve first expanding the number of PSCs, before the differentiation process to generate organoids. Expanding the number of PSCs can comprise cultivating PSCs in a basal medium with the addition of about 10-100 ng/mL FGF2 and optionally 10 μM Y27632.

In various embodiments, the step of culturing a quantity of cells includes growing the cells in a cell culture medium. The cell culture medium comprises a basal medium, and optionally with the presence of any of the growth factors or small molecules described above. A basal medium may comprise fetal bovine serum (FBS) or retinoic acid; or one of those media known in the art. In some embodiments, the basal medium in one or more steps of culturing is supplemented with a high molecular weight polymer such as methylcellulose, to increase the viscosity of the medium thereby reducing vibration and imaging artifacts. Exemplary culture mediums include for example, but are not limited to, STEMFIT medium, Roswell Park Memorial Institute (RPMI) 1640 Medium, Dulbecco's modified eagle medium (DMEM), Hank's balanced salt medium, Glasgow minimum essential medium, Ames medium, Click's medium, nutrient mixtures HAM F-10 and HAM F-12, Advanced RPMI, Apel, DMEM:F I 2. In some embodiments, CHIR, FGF9 and Actin are independently included in a cell culture medium. In one aspect, CHIR, Actin and FGF9 are added sequentially, which may be separated by 1 day, 2 days, 3 days, 4 days, 5 days, 6 days or 7 days apart between any two of the adjacently added agents. In further aspects, a cell culture medium is replaced or replenished at a user-determined interval, and agents are added in or removed from the replenishing medium such that cells are cultured with or without an agent. In some embodiments, CHIR and FGF9 are concurrently present in a cell culture medium, but not actin. In some embodiments, a cell culture medium containing FGF9, but not CHIR or activin, is used to culture the cells. In some embodiments, a cell culture medium void of FGF9, CHIR and actin is used in culturing the nephronic lineage organoids.

The cells may be cultured for at least 1 day and can be cultured indefinitely, and until the culturing is no longer desired. In some embodiments, cultures of cells form nephrotic lineage organoids in about 15 days, 16 days, 17 days, 18 days, 19 days, 20 days, 21 days, 22 days, 23 days, 24 days, or 25 days, and the cultures can be grown for 30 days or longer, e.g., the cells may be cultured for 2 months, 3 months, 6 months, 9 months, 12 months, 24 months, 30 months, 36 months, 42 months, etc. Any time periods in between the mentioned time periods for culturing the cells are also contemplated.

In various embodiments, PSCs include but are not limited to human PSCs. Exemplary human PSCs include human embryonic stem cells (ESCs) and human induced pluripotent stem cells (iPSCs). In some embodiments, the methods involve human ESCs in generating or using the nephronic lineage organoids. In some embodiments, the methods involve human iPSCs in generating or using the nephronic lineage organoids. Various embodiments provide that the quantity of PSCs in the methods are at least partially, or in a whole, with mutant polycystin 1 gene and/or mutant polycystin 2 gene. Exemplary population of cells to generate nephrotic organoids includes but are not limited to pluripotent stem cells, multipotent stem cells, progenitor cells, nephron progenitor cells, terminally differentiated cells, endothelial cells, endothelial progenitor cells, immortalized cell lines, or primary cells.

In some embodiments, the methods further involve obtaining PSCs from a subject in need of diagnosis of or treatment against polycystic kidney, and/or using the PSCs obtained from a subject in need thereof to generate a nephronic lineage organoid.

Method of Using

Imaging of organoid cultures over an extended period of time (e.g., multiple days or weeks) has been a challenge as a user's maneuvering of the culture dish causes the organoids to move. To overcome this, we used the METHOCEL medium where the METHOCEL cellulose ether adds viscosity, or a medium supplemented with a high molecular weight polymer or a thickener such as methylcellulose, hydroxyl methylcellulose, or hydroxypropyl methylcellulose (e.g., a cellulose-based thickener). Various embodiments of visualizing, tracking and/or assessing the morphology of the nephronic lineage organoids provide imaging the organoids under a microscope or camera, wherein the organoids are cultured in a viscous or gel-like medium, which in various aspects contains a polymer comprising methylcellulose, hydroxyl methylcellulose, or hydroxypropyl methylcellulose. In further embodiments, the method of visualizing, tracking and/or assessing morphology of the organoids include imaging the organoids over an extended period of time (e.g., 1 day, 2 days, 3 days, 4 days, 5 days, 6 days, 7 days, 2 weeks, 3 weeks, 4 weeks, or longer), optionally following even a single cell over multiple days in the organoid.

Various embodiments provide for methods of screening for a candidate drug for treating or reducing the incidence or severity of polycystic kidney disease, or assessing the efficacy of a molecule of interest against polycystic kidney disease, the method comprising:

contacting a molecule of interest with a nephronic lineage organoid;

measuring a level of a biomarker transcribed or expressed in the nephronic lineage organoid and/or evaluating cyst formation or progression before the contact with the molecule of interest;

measuring a level of the biomarker transcribed or expressed in the nephronic lineage organoid and/or evaluating the cyst formation or progression in the presence of the molecule of interest.

Various embodiments provide for methods of screening for a candidate drug for treating or reducing the incidence or severity of polycystic kidney disease, or assessing the efficacy of a molecule of interest against polycystic kidney disease, the method comprising:

contacting a molecule of interest with a nephronic lineage organoid; and

measuring the level of a biomarker transcribed or expressed in the nephronic lineage organoid and/or evaluating cyst formation or progression.

In some embodiments, the methods of screening involve a nephronic lineage organoids that have mutant PKD 1 gene or mutant PKD2 gene. In some embodiments, the methods of screening also include comparing the measured level of the biomarker to a reference level, wherein the reference level is that of a nephronic lineage organoids generated from PSCs with normal PKD 1 gene and normal PKD2 gene.

In various embodiments, the nephronic lineage organoid is a product by the generation process described herein. In some embodiments, the nephronic lineage organoids in the screening methods are generated from PSCs obtained from a subject in need of diagnosis of or treatment against polycystic kidney.

One or more gene/proteomics analysis can be performed with the nephronic lineage organoids in the screening methods. In some embodiments, the steps of measuring a level of a biomarker comprises genetic and/or proteomics analysis of the organoids. In some embodiments, the genetic and/or proteomics analysis includes measuring the level of, or the biomarkers measured in the screening methods include, yes-associated protein 1 (YAP1), a signal transducer and activator of transcription 1 (STAT1), polycystin1 (PKD1), polycystin2 (PKD2), hepatocyte nuclear factor 4 alpha (HNF4A), hepatitis A virus cellular receptor 1 (Havcr1), secreted phosphoprotein 1 (SPP1), tumor necrosis factor receptor superfamily member 12A (TNFRSF12A), or a combination thereof.

In some embodiments, at least some of the quantity of PSCs to generate the nephronic lineage organoid comprises mutant polycystic 1 gene and/or mutant polycystic 2 gene, and wherein the molecule of interest can be identified as a candidate drug for treating, reducing the incidence/likelihood or severity of polycystic kidney disease, or as having efficacy against the disease, when:
a) mRNA and/or protein level of HNF4A is increased in the presence of the molecule of interest, compared to that before the contact with the molecule of interest,
b) mRNA and/or protein level of HAVCR1, SPP1, STAT1, TNFRSF12A, and/or YAP1 is decreased in the presence of the molecule of interest, compared to that before the contact with the molecule of interest, or
c) the function of the mutant polycystin 1 gene or of the mutant polycystin 2 gene is restored, e.g., comparable to a normal polycystin 1/2 gene;

and wherein the molecule of interest is identified as not a candidate drug for treating, reducing the incidence/likelihood or severity of polycystic kidney disease, or as not having efficacy against the disease, when:
d) mRNA and/or protein level of Hnf4a is not increased in the presence of the molecule of interest, compared to that before the contact with the molecule of interest, or
e) mRNA and/or protein level of HAVCR1, SPP1, STAT1, TNFRSF12A, and/or YAP1 is not decreased in the presence of the molecule of interest, compared to that before the contact with the molecule of interest.

Through screening a plurality of molecules, especially protein kinase inhibitors, a few inhibitors and/or modulators of cyst formation and progression are identified, thereby treating or reducing the incidence or severity of polycystic kidney disease, including fascaplysin, an inhibitor of mitogen-activated protein kinase-interacting serine/threonine-protein kinase 1 (MNK1), PD98059, RO-3306, a dual inhibitor of CDCl₇/CDK9, 4-cyano-3-methylisoquinoline, IKK-2 inhibitor VI ((5-phenyl-2-ureido)thiophene-3-carboxamide), IKK inhibitor VII, UCN-01 (7-Hydroxystaurosporine), UCN-02 (7-epi-hydroxystaurosporine), celastrol, staurosporine, and carfilzomib. Exemplary 1VINK1 inhibitors include but are not limited to a compound of CAS no. 522629-08-9, BAY1143269, CGP57380, and eFT508. Exemplary dual inhibitor of CDCl₇/CDK9 includes PHA767491. Exemplary IKK inhibitor VII includes CAS no. 873225-46-8.

As such, various embodiments provide methods of treating or reducing the incidence or severity of polycystic kidney disease in a subject in need thereof, and the methods include administering to the subject an effective amount of an inhibitor of cyst formation and progression selected from the group consisting of fascaplysin, an inhibitor of mitogen-activated protein kinase-interacting serine/threonine-protein kinase 1 (1VINK1), PD98059, RO-3306, a dual inhibitor of CDCl₇/CDK9, 4-cyano-3-methylisoquinoline, IKK-2 inhibitor VI ((5-phenyl-2-ureido)thiophene-3-carboxamide), IKK inhibitor VII, UCN-01 (7-Hydroxystaurosporine), UCN-(7-epi-hydroxystaurosporine), celastrol, staurosporine, and carfilzomib. In some embodiments, the methods of treating do not include administering Tolvaptan to the subject.

Further embodiments provide any of the methods include selecting a subject diagnosed as having polycystic kidney(s), with polycystic kidney disease, or suffering from the disease, for generating a nephronic lineage organoid ex vivo, screening for a drug with the organoid, or treatment.

Additional embodiments provide for methods of treating, reducing the likelihood or severity of autosomal dominant polycystic kidney disease (ADPKD) in a subject in need thereof, which include screening for an agent for treating, reducing the incidence or severity of polycystic kidney disease according to the method described above, wherein the nephronic lineage organoid is optionally generated from a quantity of PSCs obtained from the subject; and administering to the subject an effective amount of the agent, said agent is selected from the group consisting of fascaplysin, an inhibitor of mitogen-activated protein kinase-interatcting serine/threonine-protein kinase 1 (MNK1), PD98059, RO-3306, a dual inhibitor of Cdc7/Cdk9, 4-cyano-3-methylisoquinoline, IKK-2 inhibitor VI ((5-phenyl-2-ureido)thiophene-3-carboxamide), IKK inhibitor VII, UCN-01 (7-Hydroxystaurosporine), UCN-02 (7-epi-hydroxystaurosporine), celastrol, staurosporine, and carfilzomib. In some embodiments, only the agent(s) screened to reduce, inhibit or prevent cyst formation/progression in the organoids are administered to the subject to treat, or reduce the incidence or severity of polycystic kidney disease. In some embodiments, agents that are screened to be ineffective in reducing, inhibiting or preventing cyst formation/progress in the organoids are not administered to the subject. Systems

Various embodiments provide for a quantity of organoids prepared by the generation methods described herein.

Various embodiments provide for a plurality of organoids, wherein an organoid comprises one, two, three, four, five, or all six of:
a quantity of nephrons, nephron progenitor cells, or both;
a quantity of interstitial cells,
a quantity of endothelial cells,
a quantity of neuron-like cells that express NEUROD1 and NEUROG1,
a quantity of neural crest-like cells that are positive for SOX/0, and
a quantity of muscle-like cells that are positive for TNNI1 and ACTC1.

In some embodiments, an organoid contains a trace quantity of human pluripotent stem cells, e.g., in a number amount from 0.1% up to no more than 1%, 2%, 3%, 4%, 5%, 6%, 7%, 8%, 9%, or 10% relative to the total number of cells in the organoid.

In some embodiments, an organoid has strong nephrogenic signatures, i.e., expressing PAX2, PAX8 or MAFB. In some embodiments, an organoid comprises interstitial cells which expressing PDGFRA but not nephrogenic genes. In some embodiments, an organoid comprises SOX/7+ CDH5+ endothelial cells.

In some embodiments, at least 10%, 20%, 30%, 40%, 50%, or 60% of the quantity of nephronic lineage organoids have cysts or develops cysts, within about 10, 11, 12, 13, 14, 15, 16, 17, 18, 19, 20, 21, 22, 23, 24, 25, 26, 27, 28, 29, or 30 days from initial differentiation of PSCs with the small molecule.

Various embodiments provide a system for generating the nephronic lineage organoid, and the system comprises:
human pluripotent stem cells (PSCs),
a growth factor comprising fibroblast growth factor (FGF) 9,
another growth factor comprising activin,
a small molecule comprising CHIR99021,
a micro-well plate,
optionally a culture medium comprising methylcellulose, and
instructional material.

In some embodiments of the system, human ESCs are included. In other embodiments of the system, human iPSCs are included. In some embodiments of the system, the growth factors comprise FGF9 and activin, each in a separate container. In some embodiments of the system, a small molecule comprising CHIR99021, or another GSK3 inhibitor, is included, one in a separate container. In some embodiments of the system, a micro-well plate that offers a low binding surface is included, which can be an EZSPHERE plate. In other embodiments of the system, a micro-well plate is surface treated with a low-binding coating, e.g., a bound hydrogel layer that inhibits cellular attachment and minimizes protein absorption.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

We have generated PKD2 mutant human ESCs which form cystic kidney mini-organoid cultures of approximately 1000 cells in EZsphere plates. We have employed a 3D culture system to emulate cystic structures in vitro and analyzed cyst initiation. This system was developed by culturing polycystin2 mutant and normal organoids modified to report on development of podocytes in cell culture medium with methylcellulose. In this system, the methylcellulose reduces organoid motility and maintains 3D structure. Together, our new mini organoids 3D culture system has great potential to become a new human model of PKD to assess the function of polycystins at the earliest disease stage.

Imaging of PKD organoids shows tubule to cyst transitions. In certain depictions herein, 4-Reporters organoid embedded in methylcellulose medium on Day 13 and cultured in the medium for 7 days. In certain depictions herein, PKD organoids embedded and cultured in methylcellulose medium. Arrowheads indicate cysts. In certain depictions herein, the whole organoid surface area relative to that at day 13 (normalized area) of all organoids with surface tubules from a single well. Each line represents the average from 4-Reporters (n=8) and PKD (n=5) (mean±s.e.m). In certain depictions herein, Images showing time courses of representative organoids in methylcellulose medium.

### Example 2. Methods of Generating and Uses of Organoids

1) Miniature Kidney Organoid Cultures

a) hPSC Maintenance

GELTREX-Coated Plate Preparation

For each 6-well or 12-well plate, 12 ml of DMEM/F12 (Life Technologies, 11320-033) was aliquoted into a 50-ml conical vial on an ice beaker. 120 μl of GELTREX was added to DMEM/F12 to make the 1% GELTREX mix. 10-ml serological pipette was used to mix the GELTREX solution thoroughly. 2 ml of 1% GELTREX was pipetted into each well of a 6-well plate (or 1 ml/well for a 12-well plate). The GELTREX plates were incubated at 37° C./5% CO₂ overnight before use.

hPSC Expansion and Maintenance

hPSCs were thawed in STEMFIT media (Ajinomoto, ASB01-R) supplemented with 100 ng/ml of FGF2 (R&D, 273-F9) and 10 μM Y27632 (Tocris, 1254) on 1% GELTREX-coated plates (ThermoFisher, A1413302). When the cells reached 70-80% confluency (1-2 days), they were passaged into a 12-well plate at 6,000 cells/well seeding density in STEMFIT media+100 ng/ml of FGF2+10 μM Y27632. The media was changed 48 hours later to STEMFIT media+100 ng/ml of FGF2 to expand the cells, and was replenished every 2 days afterward for maintenance. Each well of hPSCs was frozen in 1 ml of 10% DMSO/90% fetal bovine serum (FBS) (Genesee Scientific, 25-550) mix when the cells reach 70-80% confluency.

b) Directed Differentiation to Generate Miniature Kidney Organoids

The differentiation protocol was developed based on a protocol described in Morizane and Bonventre, 2017 and Morizane et al., 2015, which are incorporated by reference. Each biological replicate was generated from a distinct hPSC frozen vial. When the cells reached 70% confluency after thawing, they were dissociated using ACCUTASE (Gibco, A1110501), and seeded on 12-well plates at 6,000 cells/well in STEMFIT media+100 ng/ml of FGF2+10 μM Y27632. The media was changed 2 days later to STEMFIT media+100 ng/ml of FGF2 for cell expansion. 3 days after seeding, the cells were maintained in STEMFIT media+10 ng/ml of FGF2 until they were ready for differentiation.

As the cells reached 60% confluency, the differentiation procedure was started (adapted from the protocol by Morizane et al., 2015): 4 days of 8 μM CHIR99021 treatment (Sigma Aldrich, SML1046), followed by 3 days of 10 ng/ml Activin A incubation (R&D, 338-AC-050), and 1 day of 10 ng/ml FGF9 incubation (R&D, 273-F9). At day 8, the cells were dissociated using TrypLE dissociation enzyme (Gibco, 12563011), and the cell number was acquired. 600,000 cells were seeded per well of the 12-well EZSPHERE plate (Nacalai USA, TCI-4815-9035P-50P) to achieve ˜400 mini-organoids/well and 1,500 cells/organoid in 3 μM CHIR and 10 ng/ml FGF9. At differentiation day (dd) 10, the media was switched to basal differentiation media+10 ng/ml FGF9. From dd13 to dd28, the aggregates were maintained in basal media. In all differentiation steps, the basal differentiation media, which was composed of Advanced RPMI 1640 (Gibco, 12633020)+1× Glutamax (Gibco, 35050079)+1% Penicillin-Streptomycin (Invitrogen, 15070063), was used.

H9 human embryonic stem cell (hESC) line (female) was obtained from WiCell (WA09). The MAFB-P2A-eGFP hESC line was generated on the background of the H9 line as described in Tran, et al., 2019.

c) Generation of PKD1 and PKD2 Mutant Alleles

The PKD2−/− H9 line was generated using CRISPR-Cas9 technology. The gRNA (5′-CCCGGATGATGTCACAGCTCTTC-3′; SEQ ID NO:1) and Cas9 protein were delivered into the H9 cells via electroporation (Thermofisher MPK5000S). The targeted hESCs were then dissociated into single cells using ACCUTASE (Gibco, A1110501) were seeded on a GELTREX-coated 96-well plate at 1 cell/well density, in mTeSR (StemCell Technologies, 85850) supplemented with 10 μM Y27632 (Tocris, 1254). Single cell-derived colonies were expanded and validated by Sanger sequencing or qPCR for genotyping as described below.

The PKD1−/− 5-target H9 line was generated using CRISPR-Cas9 technology on the background of the 5-target (5-T) H9 line. The 5-T line is a fluorescent reporter line generated on the H9 line background to visualize different components of the nephron, but in this study, we described its use as the isogenic control in modeling polycystic kidney disease driven by PKD1−/− mutation. The generation of PKD1−/− was similar to PKD2−/− as described above, with the gRNA sequence as follow: 5′-TGGCAACGGGCACTGCTACC-3′ (SEQ ID NO:2).

Genotyping PKD1−/− Line

Single cell-derived clones of 5-T PKD1−/− hESC were expanded, and their genomic DNA was extracted using the DNeasy Blood & Tissue Kit (Qiagen, 69504). CRISPR-Cas9 targeted regions were amplified using the Q5 High-Fidelity 2× Master Mix (primers: 5′-TCCAGATGGGGCAGAGCCTG-3′ (SEQ ID NO:3) and 5′-CCTCCTTCCTCCTGAGACTC-3′ (SEQ ID NO:4)), and then cloned into the pCR™-Blunt II-TOPO™ vector using the ZERO BLUNT TOPO′ PCR Cloning Kit (Invitrogen K280002). The inserted TOPO plasmids were expanded and examined with Sanger sequencing to validate CRISPR-Cas9-induced mutations.

Genotyping PKD2−/− Line

To genotype the CRISPR-Cas9-mediated PKD2−/− mutated hESC clones, we performed quantitative PCR (Luna Universal qPCR Master Mix, New England BioLabs, M3003L) of PKD2 mRNA expression with primers specific to the sequence targeted by the gRNA (primer sequences: 5′-ACGGGAACTGGTCACATACC-3′ (SEQ ID NO:5), and 5′-ACATCATCCGGGTGTAGTAG-3′ (SEQ ID NO:6)). qPCR was carried out on the ViiA 7 Real-Time PCR System (ThermoFisher). Clones with lower expression of PKD2 were selected for further studies.

d) Embedding of Miniature Kidney Organoids for Observation and Phenotypic Drug Screening

Preparation of Methycellulose Plates

15 g of methelcellulose powder (Sigma-Aldrich, M0512) was autoclaved in a 500-ml Erlenmeyer flask. The autoclaved methylcellulose was dissolved in 60° C. 450 ml of Advanced RPMI 1640 Medium (Gibco, 12633020). 50 ml of Advanced RPMI 1640 Medium+1× Glutamax (Gibco, 35050079) and 1% Penicillin-Streptomycin (Invitrogen, 15070063) was then added at room temperature to a final volume of 500 ml. The final stock solution was cleared by centrifugation at 4000×g for 2 hours. The clear viscous supernatant was used for the spheroid assay. For mini-organoids culture and imaging, 170 μl of 20% methycellulose stock solution+80% basal differentiation media mix was added to each well of a 96-well plate (brand).

Mini-Organoid Embedding for Spheroid Assay

At dd13, miniature kidney organoids were transferred from the EZSPHERE plates to a sterile 35-mm dish by gentle pipetting with wide-bore P1000 tips. Under a dissecting microscope, 10-12 mini-organoids in 10 μl of media were picked up using a P20 pipetman and released into a well of the methylcellulose plate. A P20 pipette tip was then used to adjust the mini-organoids' positions in the well to avoid clustering. The mini-organoids then dispersed at the bottom of the well.

e) Protein Kinase Inhibitor PKD Screening of Mini-Kidney Organoid Cultures

Primary Screen:

Several commercially available libraries of pathway annotated protein kinase inhibitors were purchased from Calbiochem for screening: EMD protein kinase inhibitor 1 (Cat. No. 539744); EMD-protein kinase inhibitor-2 (Cat. No. 539745); EMD protein kinase inhibitor-3 (Cat. No. 539746); EMD protein kinase inhibitor-4 (Cat. No. 539747). All screens were carried out in the Choi Family Therapeutic Screening Facility in the Broad-CIRM center.

Protein kinase inhibitors were diluted in DMSO to make 10 μM stocks. At dd14, 20 μl of each diluted compound or DMSO was added to a methycellulose well with embedded organoids (180 μl of media) to achieve a final concentration of 1 μM. The plates were loaded on the ImageXpress Micro System for live imaging right after compounds were loaded. The imaging was performed using the “Standard” algorithm, at 4× magnification, 2 camera binning, with laser-based and image-based focusing enabled, and well-to-well autofocus was set to “focus on plate bottom and well bottom”. To avoid observer bias, we performed blinded experiments in which the compound maps were not revealed to the researcher who read the ImageXpress results until all analyses were complete.

Scoring Phenotypes:

We categorized the outcomes of compound treatments into 3 groups: 1) “non-hit” wells were those that still had cyst formation at dd20 (cyst area ≥30% organoid size), 2) “hit” wells included those that had zero cyst formed and still contained visible epithelial structures, and 3) “non-specific hit” (NS hit) wells were those without cyst formation and no visible epithelial structures due to cell death.

Secondary Screen:

Compounds that were scored as true “hits” from the primary screen were selected for validations in both PKD1−/− and PKD2−/− mutant organoids. The compounds were diluted in DMSO to 1, 10 or 100 μM via serial dilution to make the working stocks. At dd14, 20 μl of each diluted compound or DMSO was added to a methylcellulose well with embedded organoids (180 μl) to achieve a final concentration of 0.1, 1 or 10 μM. The plates were imaged for 7 days using the ImageXpress Micro System as described above.

Identification of Final “Hits”:

Compounds that inhibited cyst formation in both PKD1−/− and PKD2−/− mini-organoids were classified as final “hits”.

f) Chimeric Organoid Production

To generate wildtype-PKD2−/− chimera, differentiation of H9 CAGG-eGFP PKD2+/+ and H9 PKD2−/− hESC lines were initiated on the same day on separate plates. At dd8, eGFP and PKD2−/− cells were detached from 2-D culture. The cells were counted for each line, and were then combined at 1:1 ratio for reseeding on EZSPHERE plates (300,000 eGFP+ cells and 300,000 PKD2−/− cells per well). The resulted aggregates were composed of 50% mutant cells (eGFP−) and 50% wildtype cells (eGFP+). The aggregates followed directed differentiation as described above.

g) Viral Infection of PKD Cyst

1-2 mm diameter cysts were transferred into 96 well plates (1 cyst per well). The cysts were cultured in basal differentiation media. Three days after, supernatant was removed and cysts were subsequently incubated in basal media with Adenovirus for 24 h at 37° C. The genome equivalent used for the infections was 1×10⁵ IU/ml. After the incubation, cysts were washed once with basal differentiation media, and fresh basal differentiation was then used for subsequent maintenance. Cysts were harvested after different periods in culture (3,6 and 9d after incubation).

PKD2 rescue: 1-2 mm diameter PKD2−/− cysts were incubated with media supplemented with adenovirus at 1:2000 dilution for expression of mCherry (CMV-mCherry, 1×10\hat{ }10 PFU/ml) (Vector Biolabs, 1767) or human PKD2 (CMV-mCherry::CMV-PKD2, 3.1×10\hat{ }10 PFU/ml) (Vector Biolabs, 2001) for 24 h. Cysts were then washed, fixed and collected for analyses as described above.

h) EdU Labeling of PKD Cyst

1-2 mm cysts were cultured in basal differentiation media for 8 days. Click-iT Edu Cell Proliferation Kit for Imaging, ALEXAFLUOR™ 647 dye (Invitrogen, C10640) was used to examine proliferation of cystic cells. Cysts were cultured in basal media supplemented with 10 μM of EdU for 24 hours. They were then washed three times with 1×PBS and collected for a 10-minute 4% PFA fixation. After that, cysts were rinsed once with PBS, and EdU was detected following the manufacturer's protocol. Immunofluorescence analysis was then performed on the EdU stained samples using primary antibody against ZO-1 (ThermoFisher, 33-9100), followed by washes and secondary antibody incubation (procedure described below). DNA was counterstained with Hoechst 33342 (ThermoFisher, H3570).

2) Analysis of Mini-Organoid System

a) RNA-Seq and Single Cell RNA-Seq Analysis

About 300 miniature organoids were collected at dd8, dd10, dd14, dd16 and dd28 for scRNA-seq. The organoids were dissociated using 7.5 mg/ml Bacillus licheniformis cold active protease (Creative Enzymes, NATE-0633) mixed with 10 mg/ml collagenase type 2 (Worthington, #LS00417) and 125 U/ml DNase I (Worthington, #LS002058) in DPBS (150 μl) at 12° C. for 20 min. The digestion mix was mixed twenty times with P-1000 wide-bore pipette tips. The dissociation reaction was terminated by mixing with 150 μl of 20% fetal bovine serum in DPBS. The cells were filtered through a pre-wetted 40-μm strainer (Falcon), and 1 ml of DPBS was used to wash the cells off the strainer. The 1.3 ml of dissociated cell mix were combined with 3 ml of AutoMACS Running Buffer (Miltenyl Biotec, 130-091-221) and were pelleted at 1250 rpm at 4° C. The cell pellet was then resuspended in 350 μl of AutoMACS Running Buffer, with 14 uM DAPI and 5 uM DRAQS added freshly. The cells were subjected for fluorescence-activated cell sorting (FACS) to select for single live cells (DAPI-DRAQS+). We used the 10× Genomics Chromium Single Cell 3′ GEM, Library & Gel Bead Kit (10× Genomics, PN-1000075) to capture and process single cells for transcriptomic profiling. After being recovered from the emulsion, cDNA was cleaned-up, amplified by PCR, examined on a 4200 Tape station (Agilent) for yield assessment, and then processed into barcoded library for Illumina sequencing. Paired-end sequencing on the Illumina HiSeq 4000 platform was performed using the HiSeq 3000/4000 SBS PE clustering kit (PE-410-001) and 150 cycle flow cell (FC-410-1002). From fastq files, quality control, alignment to reference genome (hg38) and generation of count tables of the five libraries were done using CellRanger 3.1 (10× Genomics).

The Seurat 3.0 package was used for scRNA-seq analyses (Stuart et al., 2019). The five datasets merged using the merge function. To filter out low-quality cells, we kept cells that had more than 500 and fewer than 5,500 features, fewer than 20,000 RNA counts, and less than 35% mitochondrial gene content. The merged data was log-normalized using the NormalizeData function. To scale and center genes in the dataset, the ScaleData function was applied. 2000 variable genes were determined using the Find VariableFeatures function. The RunPCA was applied to calculate principle components (PCs), and 40 PCs were used to determine neighbor cells and cluster assignment (using the FindNeighbors and FindClusters functions). The UMAP reduction was calculated using RunUMAP to determine UMAP embedding. Differentially expressed genes of each cluster were found using the FindAllMarkers function.

The in vivo datasets of human week 17 fetal kidney from our 2019 study (Tran et al., 2019; GEO accession number GSE124472) were used for comparison with the scRNA-seq profiles of the in vitro derived nephrogenic cell subset. After the nephrogenic cells were subset from the week 17 datasets, the in vitro and in vivo nephrogenic cells were merged and integrated using scTransform. The merged dataset was first split based on in vitro or in vivo origin of the cells. scTransform was performed on each origin using 10,000 variable features. To prepare for integration, integration features and anchors were determined using SelectlntegrationFeatures, PrepSCTlntegration and FindlntegrationAnchors and 20 PCs. The two origins are then integrated using IntegrateData. RunPCA was then used to calculate PCs, and 40 PCs were used for neighbor and cluster finding as described above. Cell embeddings were presented in UMAP reduction.

b) Histology

Mini-organoids were fix in 4% paraformaldehyde for 10 minutes at 4° C. temperature and were washed three times in 1×PBS. Samples were then transferred to an embedding mold with 15% sucrose/7.5% gelatin in PBS and incubated in the gelatin solution at 37° C. until the organoids sink. The mini-organoids in gelatin solution was then frozen in a dry ice/ethanol slurry. Samples were then stored at −80° C. until cryosectioning.

Frozen sections were warmed up room temp for 10 minutes before the staining procedure. 1× Citrate Buffer pH 6.0 (Sigma) was used for antigen retrival in a pressure cooker. The slides were then washed with water and air dried for 5 min. 1.5% Seablock (ThermoFisher) in PBS+0.25% TritonX block buffer was applied on the tissue for 1 hour at room temperature for blocking. The slides were then incubated with primary antibody mixture (diluted in block buffer) at 4° C. overnight. Primary antibodies used in the study are listed as follow: WT1 (abcam, ab89901, 1:5000), JAG1 (R&D, AF599, 1:300), LAMB1 (Santa Cruz, sc-33709, 1:50), SOX9 (abeam, ab185230, 1:1000), HNF4A (R&D, MAB4605, 1:500), CUBN (R&D, AF3700, 1:500), SLC12A1 (Sigma, HPA018107, 1:500), LTL (Vector Laboratories, FL-1321, 1:300), SLC3A1 (Sigma, HPA038360, 1:500), NPHS1 (abeam, ab136927, 1:5000), POU3F3 (ThermoFisher, PA5-64311, 1:500), MAFB (R&D, MAB3810, 1:500), PAX8 (abeam, ab189249, 1:1000), CDH1 (Biosciences, 610182, 1:300), PAX2 (R&D, AF3364, 1:500), GATA3 (R&D, AF2605, 1:300). We used secondary antibodies conjugated with ALEXAFLUOR 488, 555, 594, and 647 (diluted to 1:1000 in block buffer) purchased from Molecular Probes. To stain the nuclei, slides were treated with 1 mg/ml Hoechst 33342 (Molecular Probes) in PBS for 5 min. ProLong Gold Antifade Reagent (Life technologies) was applied on the tissue for mounting, and images were acquired at 40× using the Leica SP8 confocal microscope.

d) RNA Extraction, cDNA Synthesis and Quantitative Polymerase Chain Reaction

About 200 mini-organoids were collected for transcriptional analyses for each time point. The RNeasy Micro Kit (Qiagen, 74004) was used for RNA extraction following the manufacturer's protocol. cDNA was synthesized from 200 μg of RNA for each sample using the SuperScript IV VILO Master Mix with ezDNase enzyme (Invitrogen, 11766050).

Quantitative polymerase chain reaction (qPCR) was performed using the Taqman Fast Advanced Master Mix (ThermoFisher, 444557) following the manufacturer's instruction on the ViiA 7 Real-Time PCR System (ThermoFisher). The following probes from ThermoFisher were used for transcriptional analyses: WT1 (Hs01103751_m1), MAFB (Hs00534343_s1), PAX2 (Hs01057416_m1), HNF4A (Hs00230853_m1), GATA3 (Hs00231122_m1), SLC3A1 (Hs00942976_m1), SLC12A1 (Hs00165731_m1) and SLC12A3 (Hs01027568 _m1).

### Example 3. Development and Validation of a Reproducible Miniature Kidney Organoid System Generating Nephron-Like Structures for Systematic Screens

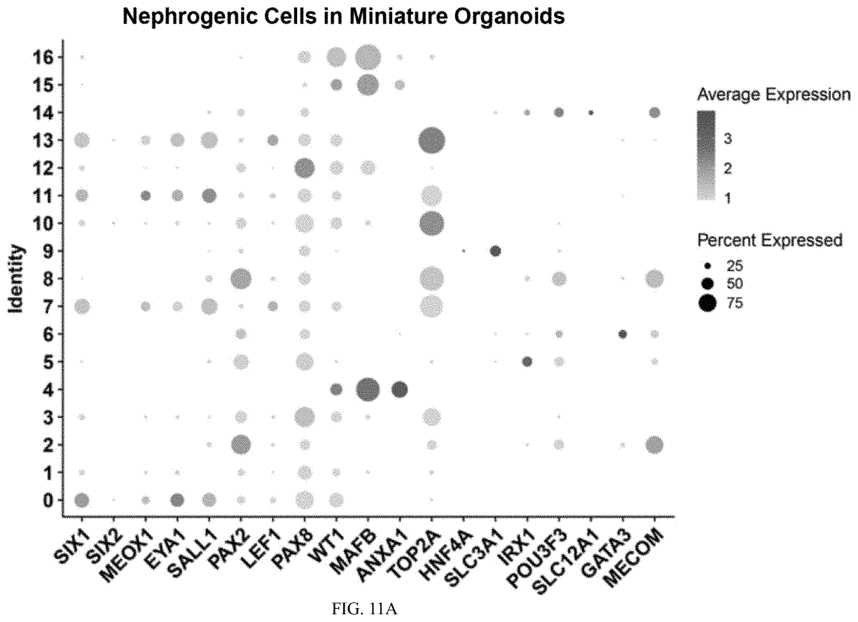

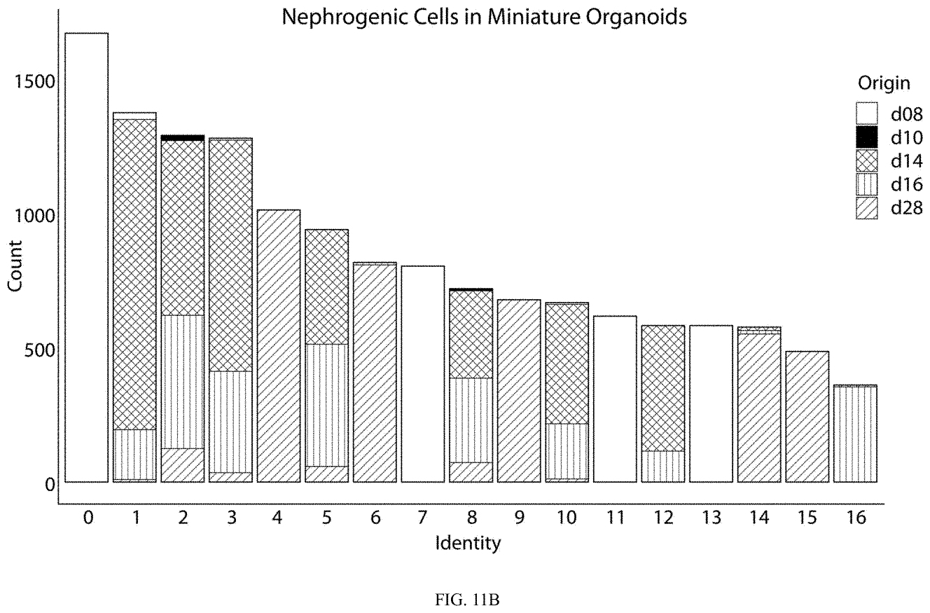

To achieve large-scale production of 3-D kidney organoids, we utilized EZSPHERE 12-well plates, which were constructed using laser-based microfabrication to contain uniform microwells of 800-μm diameter and 400-μm depth (Sato et al., 2016). At dd8, cells were dissociated into single cells, and 600,000 cells were reseeded in each well of the EZSPHERE plate to produce about 400 miniature 3-D aggregates with 1,500 cells per aggregate (FIG. 7). With 400 engineered wells in each of the 12 culture wells, the system allows for the generation of up to 4,800 mini-organoids per dish. We directly visualized and monitored nephron development. we used a MAFB-P2A-eGFP H9 hESC line to visualize the formation of podocyte-like cells in the mini-organoids. The emergence of eGFP+ cells at day 14 of differentiation, agreeing with previous observation in the 100,000-cell kidney organoids generated in 96-well plates (we called these “maxi-organoids” herein). At dd25, each miniature organoids comprised 1-2 eGFP+ clusters, suggesting 1-2 nephron-like structures formed in each organoid (FIG. 8).

Next, we characterized the developmental program and cellular composition of the miniature kidney organoids, applying a current understanding of human nephrogenesis from a series of studies in the McMahon laboratory. Transcriptional profiling using qPCR highlighted robust upregulation of nephrogenic signatures (including WT1, PAX2, MAFB, HNF4A, GATA3, SLC3A1, SLC12A1 and SLC12A3) over the differentiation time course in MAFB-P2A-eGFR organoids. Immunofluorescent analyses using antibodies specific for each of these proteins demonstrated the presence of each protein within the organoids. Furthermore, the distribution of proteins demarcated specific nephron cell types or segments showed a similar spatial relationship to that of the nephron in the human kidney. These findings corroborate key predictions from the transcriptional profiling. At dd13, we documented the presence of CDH1+ epithelial renal vesicle-like structures with WT1-high and WT1-low domains, which indicates early nephron polarization-like process. Dd13 also marked the emergence of cells with signatures of proximal nephron segment precursors (MAFB+ or WT1+), medial precursors (MAFB−/SOX9−/JAG1+, or HNF4A+), and distal nephron precursors (SOX9+ or GATA3+). At dd14 and dd16, the segmentation of developing nephron-like structures became more apparent with major proximal, medial and distal precursor domains. Additionally, we also detected the presence of micro-domains in the dd25 in vitro derived nephrons: WT1+ HNF4A+, HNF4A+POU3F3+ and GATA3+POU3F3+. Though these segments have not been fate mapped in the mammalian kidney, the first two are expected to give rise to the proximal tubule segments respectively, while the last one is predicted to generate the distal connecting segment. At dd25, mature nephron-like structures were also detected: MAFB+ podocyte-like cells clustered adjacent to an epithelial CUBN+ proximal tubule-like segment, which connected to an SLC12A1+ loop of Henle-like segment. Though we noted the upregulation of SLC12A3, a distinct distal tubule segment marker at the transcriptional level, levels were too low to detect SLC12A3 protein. Collectively, the data supported the emergence of segmented nephron-like structures in the miniature kidney organoids while lacking the stereotyped morphogenesis described in human nephrogenesis.

Developmental Trajectories of Nephron-Like Cell Types

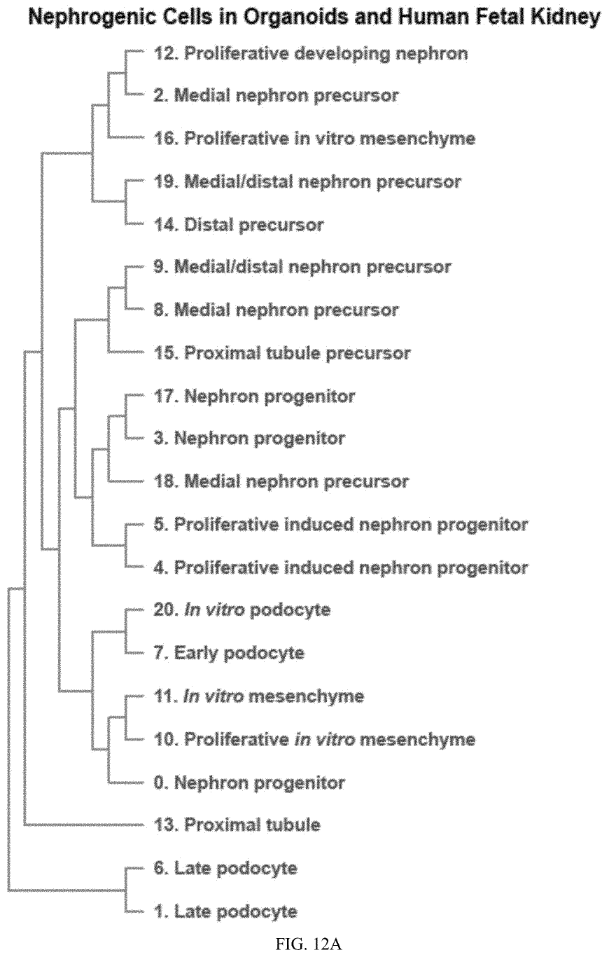

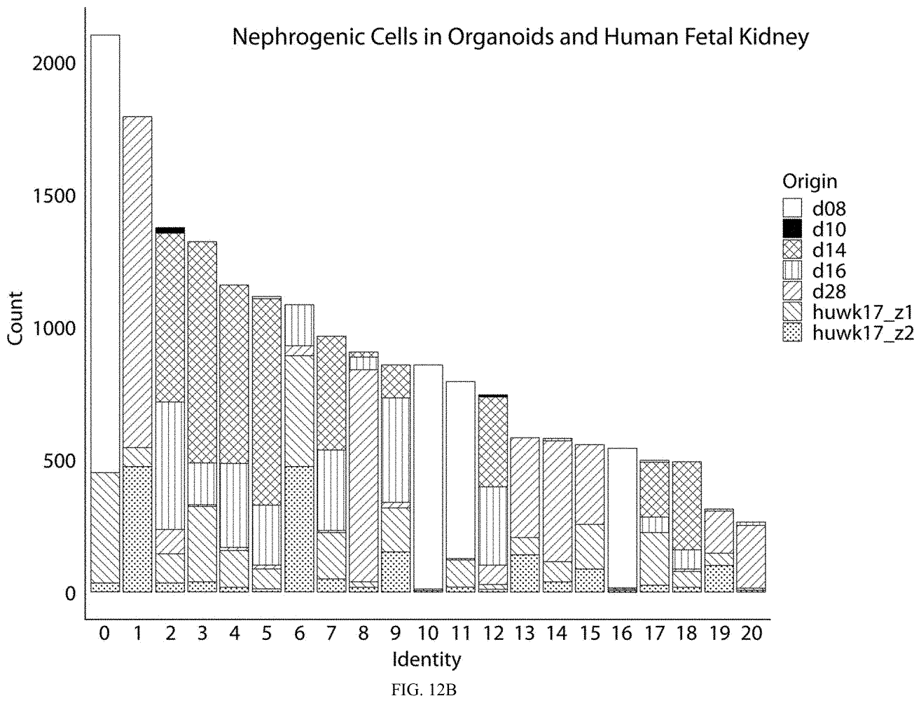

To further examine the emergence of nephron-like cells in miniature organoids, we utilized the single-cell RNA-sequencing (scRNA-seq) technology to capture single-cell transcriptomic profiles of organoids at dd8, dd10, dd14, dd16 and d2. Using the Seurat package (version 3), we selected against low-quality cells, performed log-normalization, scaled the data, determined the dimensionality, and identified “neighbors” and “clusters” using the first 40 principle components. We assigned cluster identities by applying well-established markers and documented the cellular diversity in the miniature organoids (FIGS. 9 and 10). Besides cells with strong nephrogenic signatures (expressing PAX2, PAX8 or MAFB; clusters 1, 3, 4, 5, 8, 9, 10, 19, 27, 28 and 29), the organoids were also composed of interstitial cells (clusters 2, 11, 12, 22, 23 and 24 expressing PDGFRA but not nephrogenic genes), SOX/7+ CDH5+ endothelial cells (cluster 31), neuron-like cells (clusters 13, 14, 17 and 25, identified by NEUROD1 and NEUROG1 expression), SOX/0+ neural crest-like cells (cluster 6, 7, 18, 20 and 21), muscle-like cells (TNNI1+ ACTC1+ cluster 16), and unidentified cell types (cluster 0, 15 and 26) (FIG. 10A). All groups who have performed similar detailed analysis of human kidney organoid models reported similar non-kidney cell types, independent of differences in kidney organoid generation or culture conditions.

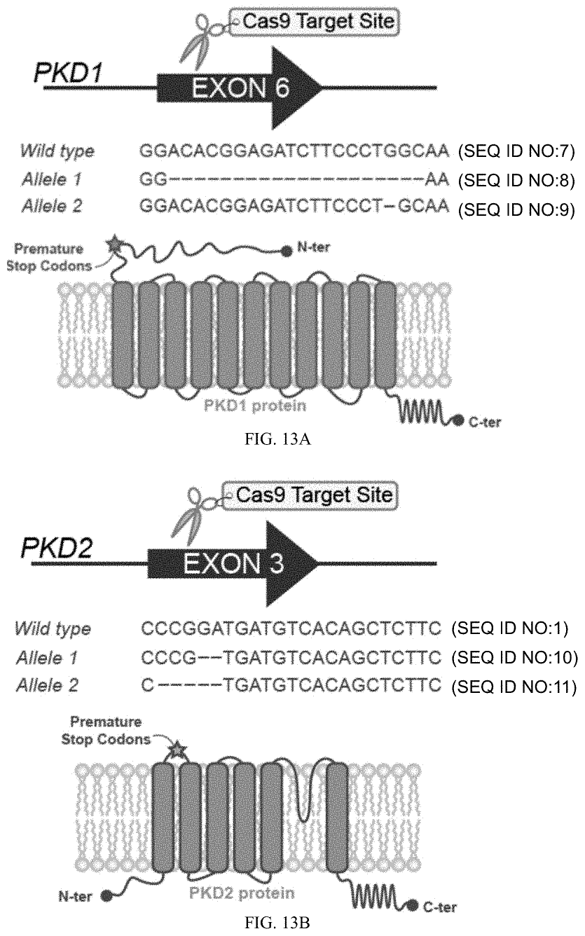

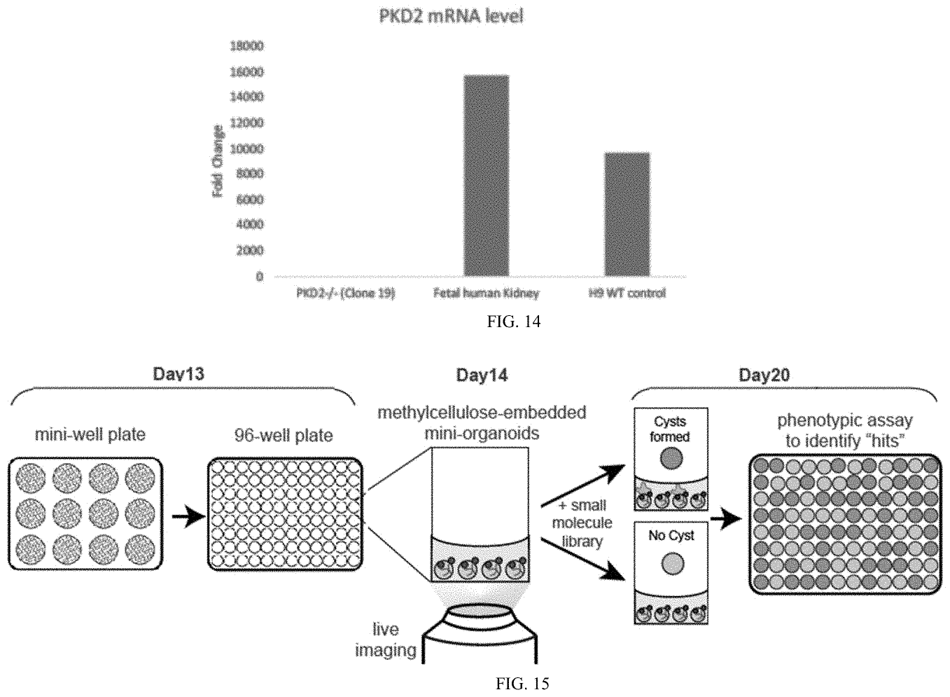

We subset 14566 cells from the nephrogenic clusters for further analyses (FIG. 10B). Re-clustering of this sub-population identified cells with strong transcriptional characteristics of induced nephron progenitors, early and late podocytes, medial and distal precursors, and proximal tubule cells (FIG. 11A). When examining the origins contributing to the various nephrogenic identities, we noticed induced nephron progenitor-like clusters were dominantly composed of day 8 cells, while day 10, 14 and 16 cells contributed to nephron segment precursors (FIG. 11B). Late podocyte-like cells and proximal tubule-like cells were detected in day 28 mini-organoids. Nonetheless, medial/distal precursor cells were also observed in organoids of this late timepoint (indicated by the absence of mature markers like UMOD and SLC12A3), indicating an incomplete maturation of nephron tubules in the in vitro system. To compare the in vitro derived cells with the human kidney, we subset 4377 nephrogenic cells from week 17 human fetal kidney datasets and merged the in vivo with the in vitro derived nephrogenic cells. Cluster identification validated the presence of nephrogenic cell types (FIG. 12A). Nephron progenitor, nephron segment precursor, late podocyte and proximal tubule identities were composed of both human fetal kidney and in vitro origins, highlighting the similarity between the in vivo and in vitro derived cells. Of note, clusters 10 and 16 (proliferative in vitro mesenchyme) and cluster 20 (in vitro podocyte) were predominantly composed of organoid cells (FIG. 12B), indicative of molecular differences in cell profiles between in vivo and in vitro derived cells.

Modeling Polycystic Kidney Diseases Using Miniature Kidney Organoids

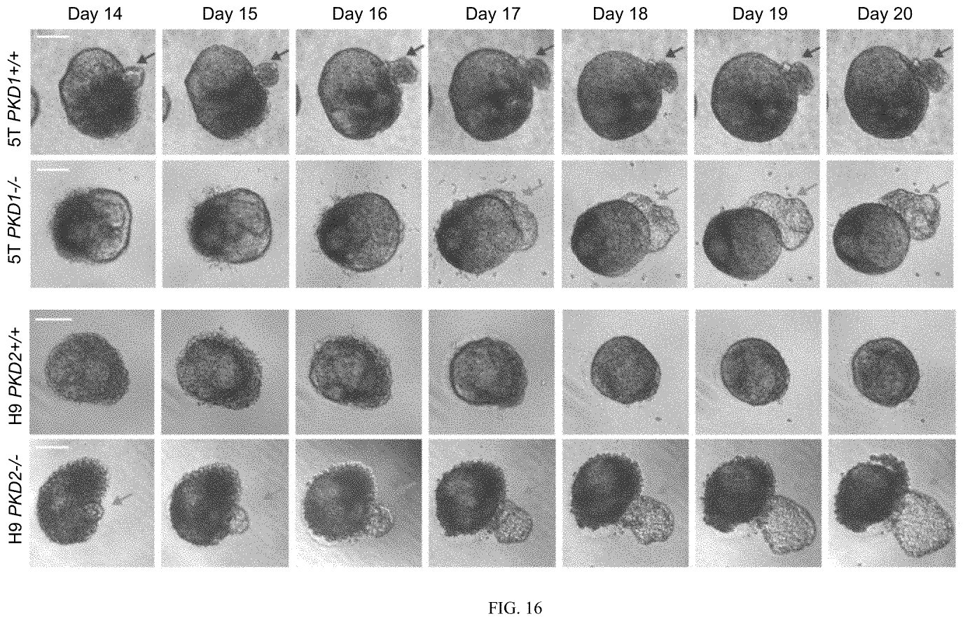

To explore the utility of the miniature kidney organoids in modeling polycystic kidney disease, we generated the PKD1−/− line (on the background of 5-T H9 hESC) and the PKD2−/− line (on the background of H9 hESC) using CRISPR-Cas9 technology (FIGS. 13A and 13B). Acknowledging the genetic complexity of the PKD1 gene, which has six known highly homologous pseudogenes, we designed a guide RNA specific to the PKD1 gene sequence but not pseudogenes:

PKD1 Ref Seq. From Location 91 to 180:

(SEQ ID NO: 12)

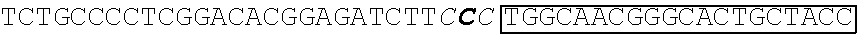

GCCTGGTGGTGGAGAAGGCGGCCTGGCTGCAGGCGCAGGAGC,

wherein region in italics indicates PAM seq., boxed region indicates gRNA, and bolded nucleic acids indicate two locations of possible gene/pseudogene variations.
Clone 1 (mutant) from location 285 to 373: is different from SEQ ID NO:12 in that clone 1 lacks the second nucleic acid within the gRNA region that is present in SEQ ID NO:12.
Clone 2 (mutant) from location 285 to 373: is the same as clone 1.
Clone 3 (mutant) from location 762 to 674: is different from SEQ ID NO:12 in that clone 3 lacks the third nucleic acid within the gRNA region that is present in SEQ ID NO:12.
Clone 4 (mutant) from location 163 to 251: is different from SEQ ID NO:12 in that clone 4 lacks the second nucleic acid within the gRNA region that is present in SEQ ID NO:12.
Clone 5 (mutant) from location 338 to 250: is different from SEQ ID NO:12 in that clone 5 lacks the third nucleic acid within the gRNA region that is present in SEQ ID NO:12.
Clone 6 (pseudogene) from location 337 to 248: is different from SEQ ID NO:12 in that the first location of possible gene/pseudogene variation of clone 6 is T, rather than C as in SEQ ID NO:12.
Clone 7 (pseudogene) from location 339 to 250: is different from SEQ ID NO:12 in that the first location of possible gene/pseudogene variation of clone 7 is T, rather than C as in SEQ ID NO:12.
Clone 8 (pseudogene) from location 338 to 249: is different from SEQ ID NO:12 in that the first location of possible gene/pseudogene variation of clone 8 is T, rather than C as in SEQ ID NO:12.
Clone 9 (pseudogene) from location 284 to 373: is different from SEQ ID NO:12 in that the first location of possible gene/pseudogene variation of clone 9 is T, rather than C as in SEQ ID NO:12.
Clone 10 (pseudogene) from location 764 to 675: is different from SEQ ID NO:12 in that the first location of possible gene/pseudogene variation of clone 10 is T, rather than C as in SEQ ID NO:12.
Clone 11 (pseudogene) from location 283 to 372: is different from SEQ ID NO:12 in that the first location of possible gene/pseudogene variation of clone 11 is T, rather than C as in SEQ ID NO:12.
Clone 12 (pseudogene) from location 764 to 675: is different from SEQ ID NO:12 in that both locations of possible gene/pseudogene variation of clone 12 are T, rather than C as in SEQ ID NO:12.
Clones 13-16 (pseudogene) from location 337 to 248: are different from SEQ ID NO:12 in that both locations of possible gene/pseudogene variation of each of these clones are T, rather than C as in SEQ ID NO:12.
Clone 17 (pseudogene) from location 338 to 249: is different from SEQ ID NO:12 in that both locations of possible gene/pseudogene variation of clone 17 are T, rather than C as in SEQ ID NO:12.
Clone 18 (pseudogene) from location 160 to 249: is different from SEQ ID NO:12 in that both locations of possible gene/pseudogene variation of clone 18 are T, rather than C as in SEQ ID NO:12.
Clone 19 (pseudogene) from location 163 to 252: is different from SEQ ID NO:12 in that both locations of possible gene/pseudogene variation of clone 19 are T, rather than C as in SEQ ID NO:12.
Clones 20, 22 and 28 (pseudogene) from location 764 to 675: are different from SEQ ID NO:12 in that both locations of possible gene/pseudogene variation of each of these clones are T, rather than C as in SEQ ID NO:12.
Clone 21 (pseudogene) from location 765 to 676: is different from SEQ ID NO:12 in that both locations of possible gene/pseudogene variation of clone 21 are T, rather than C as in SEQ ID NO:12.
Clone 23 (pseudogene) from location 765 to 676: is the same as clone 21.
Clones 24 and 30 (pseudogene) from location 286 to 375: are different from SEQ ID NO:12 in that both locations of possible gene/pseudogene variation of each of these clones are T, rather than C as in SEQ ID NO:12.
Clones 25-27, 29, 31 and 34 (pseudogene) from location 763 to 674: are different from SEQ ID NO:12 in that both locations of possible gene/pseudogene variation of each of these clones are T, rather than C as in SEQ ID NO:12.
Clone 32 (pseudogene) from location 284 to 373: is different from SEQ ID NO:12 in that both locations of possible gene/pseudogene variation of clone 32 are T, rather than C as in SEQ ID NO:12.
Clones 33 and 35 (pseudogene) from location 285 to 374: are different from SEQ ID NO:12 in that both locations of possible gene/pseudogene variation of each of these clones are T, rather than C as in SEQ ID NO:12.
Clone 36 (mutant) from location 319 to 250:

(SEQ ID NO: 13)
TCTGCCCCTCGGAACGGGCACTGCTACCGCCTGGTGGTGGAGAAGGCGGC
CTGGCTGCAGGCGCAGGAGC.

Clones 37 and 38 (mutant) from location 318 to 249: are the same as SEQ ID NO:13.

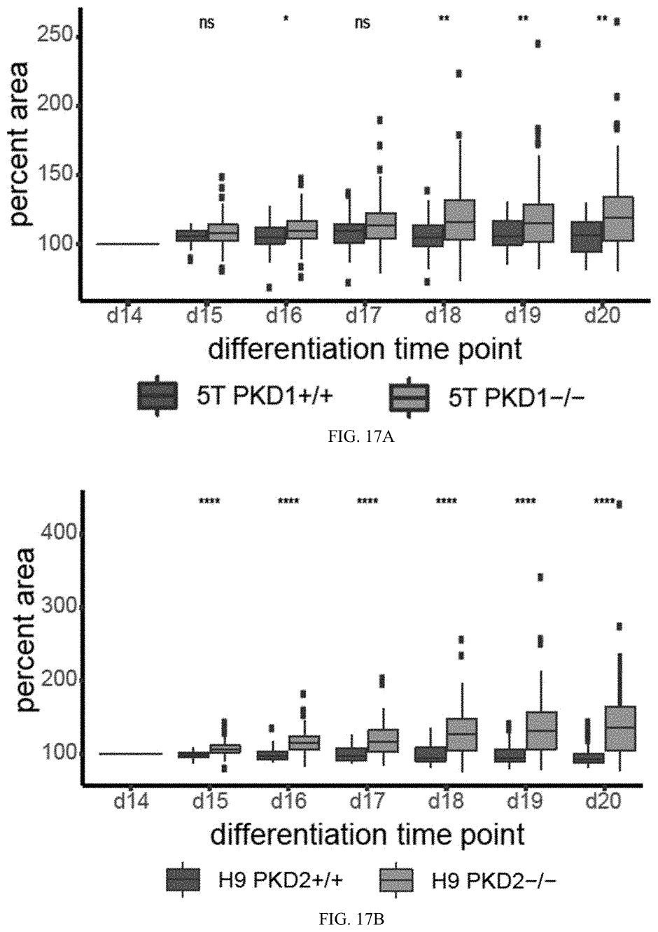

The deletion mutations at the desired loci for both PKD1−/− were validated using Sanger sequencing of DNA clones amplified from genomic DNA of PKD1−/− organoids, confirming that the CRISPR-Cas9 induced mutations indeed occurred at the PKD1 gene loci and not at pseudogenes. For PKD2 mutation, we validated the deletion by qPCR with primers distinguishing mutant and wild type mRNAs (FIG. 14). Collectively, the data substantiated the validity of our PKD1−/− and PKD2−/− hESC lines as reliable genetic tools for us to perform disease modeling studies.

PKD1−/− and PKD2−/− hESCs were differentiated alongside their isogenic controls. To examine nephrogenesis in the mutant mini-organoids, we performed transcriptional profiling qPCR and confirmed the activation of key nephron segment markers: MAFB, SLC3A1, SLC12A1 and SLC12A3 in PKD2−/− organoids, in 5-T organoids, and in 5-T PKD1−/− organoids.

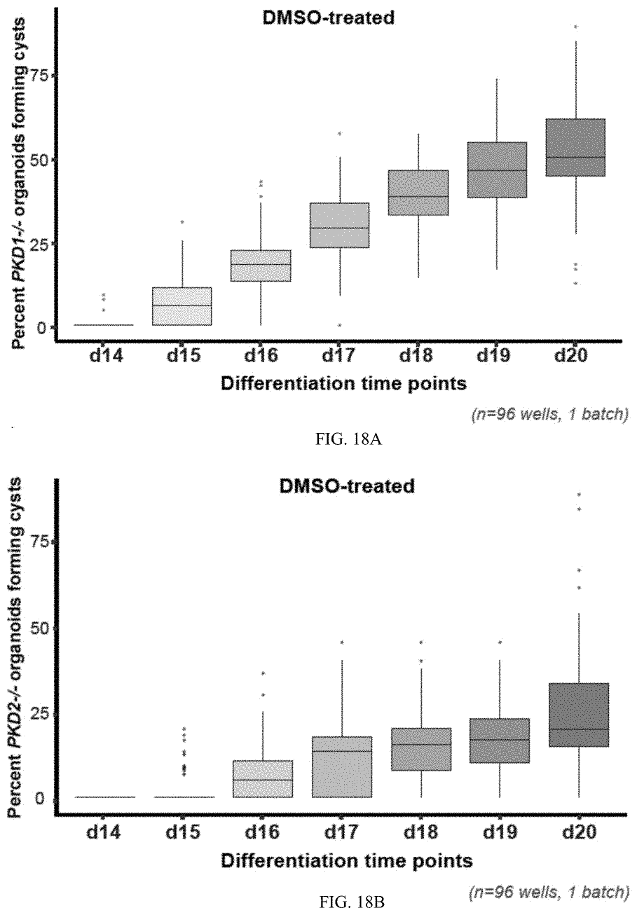

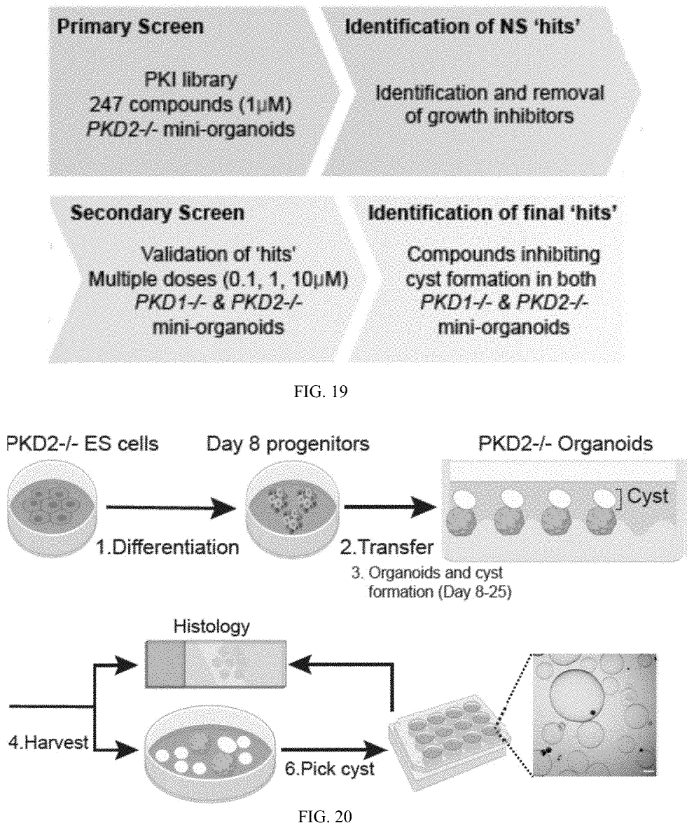

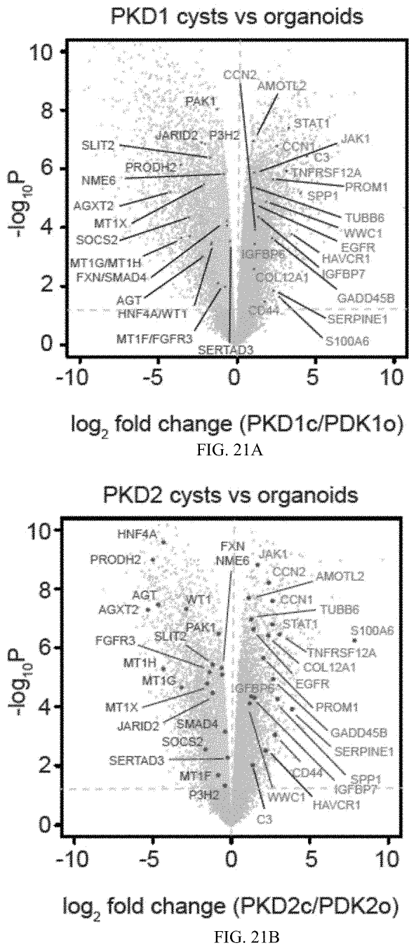

At dd13, we embedded mutant organoids and their isogenic controls in methylcellulose media and performed automated imaging every 24 hours to track their progression (FIG. 15). At dd14, PKD2−/− and PKD1−/− organoids formed protrusions that enlarged and became epithelial fluid filled cysts as early as dd15 (FIG. 16). Epithelial protrusions were found in the isogenic controls as well but they did not form cysts. As we defined a cystic structure as a clear epithelial protrusion ≥30% of the organoid size, cysts were detected with confidence at a higher rate within dd15-19 in miniature mutant organoids compared to organoids seeded at 5000 cells or 7000 cells, highlighting the advantage of our system to detect cyst initiation. We tracked 99 PKD1−/− organoids, 32 PKD2−/− organoids with their isogenic controls from dd14 to dd20, and documented significant increases in total organoid area in the mutant groups (FIGS. 17A and 17B). Immunofluorescent analysis presented the cellular diversity within the cystic epithelia, with cells resembling medial nephron segment (JAG1+, HNF4A+ or SLC3A1+) and distal nephron domain (SOX9+ or POU3F3+) contributing to the cysts' epithelial periphery.

Phenotypic Screen to Identify Protein Kinase Inhibitors Impeding Cyst Initiation

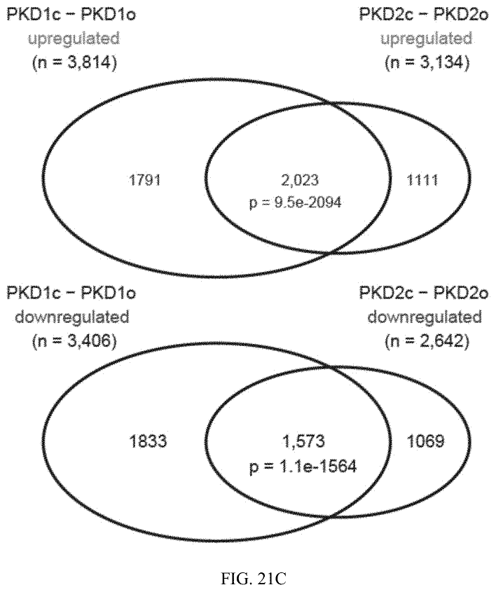

With the advantage of miniature organoid mass production, we designed a phenotypic screen workflow to find protein kinase inhibitors (PKIs) that can prevent the initiation of cysts (FIG. 15). We first identified the cyst formation rates of vehicle- (DMSO−) treated organoids. 933 PKD1−/− and 1241 PKD2−/− mutant organoids were monitored starting at dd14, and brightfield images were captured every 24 hours. At dd20, 52.3% of PKD1−/− and 25.3% of PKD2−/− organoids formed cysts in average (FIGS. 18A and 18B). We then calculated Z-scores for each time point to evaluate the feasibility of a phenotypic assay based on the DMSO treatment counts. Positive Z-scores at dd20 for both the PKD1−/− and PKD2−/− lines (0.40 and 0.37 respectively) demonstrated that positive and negative outcomes could be determined with confidence. Z-scores at dd19 for the PKD1−/− and PKD2−/− lines were 0.40 and 0.37, respectively. Z-scores at dd18 for the PKD1−/− and PKD2−/− lines were 0.27 and 0.22, respectively. Z-scores at dd17 for the PKD1−/− and PKD2−/− lines were 0.07 and −0.03, respectively. Z-scores at dd16 for the PKD1−/− and PKD2−/− lines were −0.65 and −1.60, respectively. Z-scores at dd15 for the PKD1−/− and PKD2−/− lines were −1.07 and −5.74, respectively.

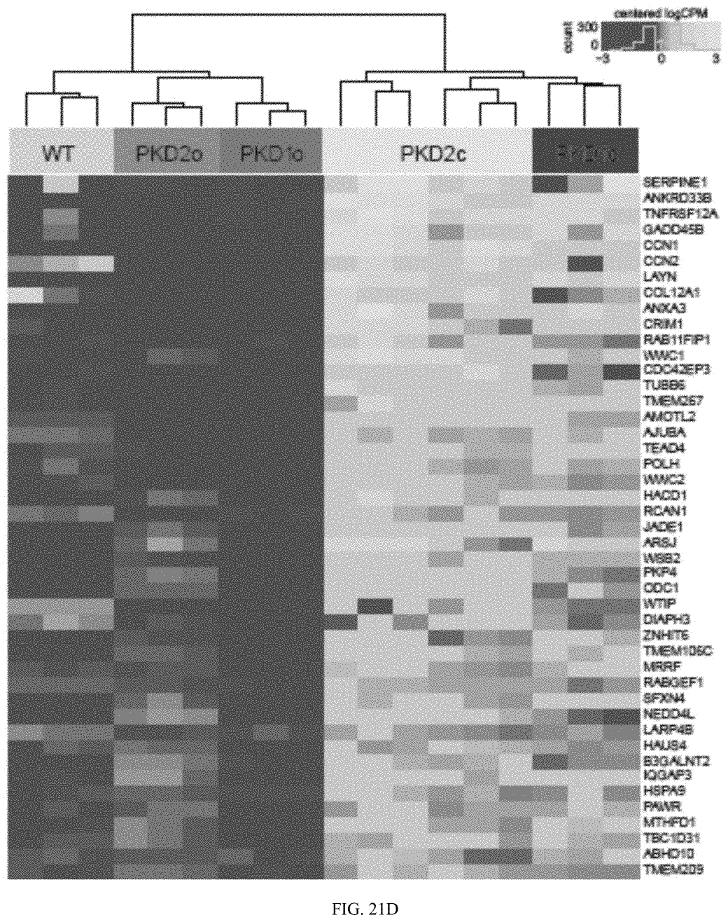

To gain new insight into pathways specifically regulating cystic growth and to validate the mini-organoid system as a robust drug screening tool, we screened a commercial library of pathway annotated protein kinase inhibitors (FIG. 19). The primary screen was performed using a library of 247 protein kinase inhibitors (PKIs) (at 1 μM) on PKD2−/− organoids, with 9 wells on 3 separate plates per compound. We categorized the dd20 outcomes into three groups: 1) “non-hit” wells were those having cyst formation at dd20, 2) “hit” wells included those without cyst formation and still containing visible epithelial structures, and 3) “non-specific hit” (NS hit) wells were those without cyst formation but no visible epithelial structures due to cell death. To increase the stringency of the screen, only compounds identified as “hits” in all 9 wells were considered true “hits” and selected for a secondary screen.

Among the 247 initial screen compounds screened on PKD2−/− organoids, 11 showed general cell growth inhibition, while nine demonstrated more specific inhibition of cystic growth. These nine “specific” inhibitors are fascaplysin (CAS no. 114719-57-2), MNK1 inhibitor (CAS no. 522629-08-9), PD 98059 (CAS no. 167869-21-8), Cdk1 inhibitor IV RO-3306 (CAS no. 872573-9308), Cdc7/Cdk9 inhibitor (CAS no. 845714-00-3), 4-Cyano-3-methylisoquinoline (CAS no. 161468-32-2), IKK-2 inhibitor VI (CAS no. 354811-10-2), IKK inhibitor VII (CAS no. 873225-46-8), and UCN-02 (CAS no. 121569-61-7).

With these initial 9 hits, control growth inhibitors and Celastrol and Tolvaptan were screened in a secondary assay of cystogenesis using both PKD1−/− and PKD2−/− organoids. Tolvaptan is the first FDA approved pharmacological treatment for ADPKD targeting cAMP levels through the inhibition of AVPR2. However, AVPR2 is restricted to non-nephrogenic lineages (the collecting system) so Tolvaptan is not expected to work on a spectrum of ADPKD patients where cysts are generated by epithelial nephron cell types, as in the mini-organoid cystic models. The anti-inflammatory compound, Celastrol (pentacyclic triterpene), has been reported to slow cyst growth and maintains kidney function in murine models of PKD though the mechanism of action is unclear.

We examined dosage-dependent responses by applying 3 different compound concentrations of each compound: 0.1, 1 and 10 μM. Celastrol was identified as a “hit” at 1 but was more broadly detrimental to cell growth at 10 μM in PKD2−/− organoids. As expected, Tolvaptan was a “non-hit” compound at all three concentrations for both PKD1−/− and PKD2−/− organoids. Compounds impeding cyst formation in both PKD1−/− and PKD2−/− mutants were listed below:

TABLE 1

| | | PKD2−/− | | | PKD1−/− | | |
|---|---|---|---|---|---|---|---|
| | Compound | 0.1 | 1 | 10 | 0.1 | 1 | 10 |
| CAS no. | Name | μM | μM | μM | μM | μM | μM |
| 34157-83-0 | Celastrol | ∘ | ▴ | ▪ | | | |
| 150683-30-0 | Tolvaptan | ∘ | ∘ | ∘ | ∘ | ∘ | ∘ |
| 112953-11-4 | UCN-01 | ∘ | ▴ | ▪ | ∘ | ∘ | ▪ |
| 121569-61-7 | UCN-02 | ∘ | ▪ | ▪ | ▴ | ▴ | ▪ |
| 62996-74-1 | Staurosporine | ▪ | ▪ | ▪ | ▪ | ▪ | ▪ |
| 868540-17-4 | Carfilzomib | ▴ | ▴ | ▴ | ∘ | ∘ | ▴ |
| 545380-34-5 | NF-κB | ▴ | ▴ | ▴ | ▴ | ▴ | ▴ |
| | activation | | | | | | |
| | inhibitor | | | | | | |
| 873225-46-8 | IKK inhibitor | ∘ | ∘ | ▴ | ∘ | ∘ | ▴ |
| | VII | | | | | | |

∘ Cysts formed; ▪ No cyst, cell growth affected; ▴ no cyst.

Among these were compounds showed to inhibit protein kinase C pathway (UCN-01, UCN-02 and Staurosporine), and those affecting the NF-κB pathway (Carfilzomib, NF-x13 activation inhibitor, and IKK inhibitor VII). Of note, Carfilzomib prevented cyst formation effectively at the lowest testing concentrations (0.1 μM) in PKD2−/− mutant organoids but only at the highest concentration for PKD1−/− cystic organoids. In contrast, the NF-κ13 activation inhibitor 545380-34-5 was highly effective inhibiting cystic growth at 0.1 μM in both PKD1−/− and PKD2−/− mutants. The hit compound overlaps between PKD1−/− and PKD2−/− strongly support the validity of our assay and point to protein kinase C and NF-κB pathways as pathways for further analysis. Further, the strong inhibitory effects of the NF-κB activation inhibitor 545380-34-5 support a more focused follow up of compounds with similar structures in our in vitro assays and animal model systems.

We followed up on the activation of the NF-κB pathway in our in vitro system as well as in the CAGG-CreER™; Pkd2^{fl/fl} mouse model, which carried PKD2 deletion upon tamoxifen injection at postnatal day 7 and 14, after tamoxifen induced removal of Pkd2 activity. RelB, a transcriptional mediator of non-canonical NF-kB signaling was activated in early cysts as showed by immunofluorescence.

RNA-Directed Screening of PKD Mutant Cysts

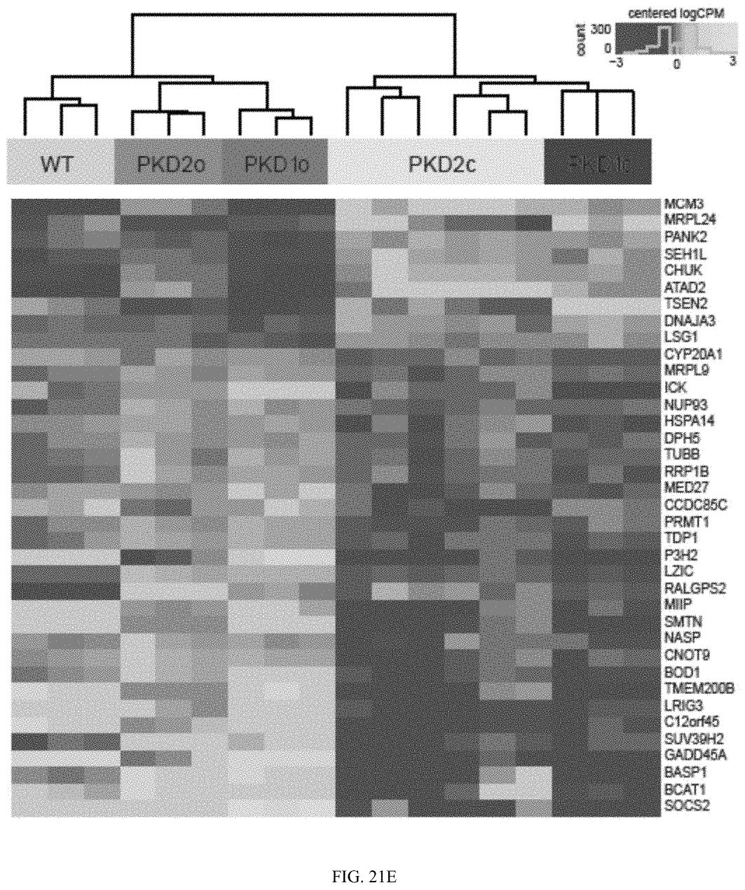

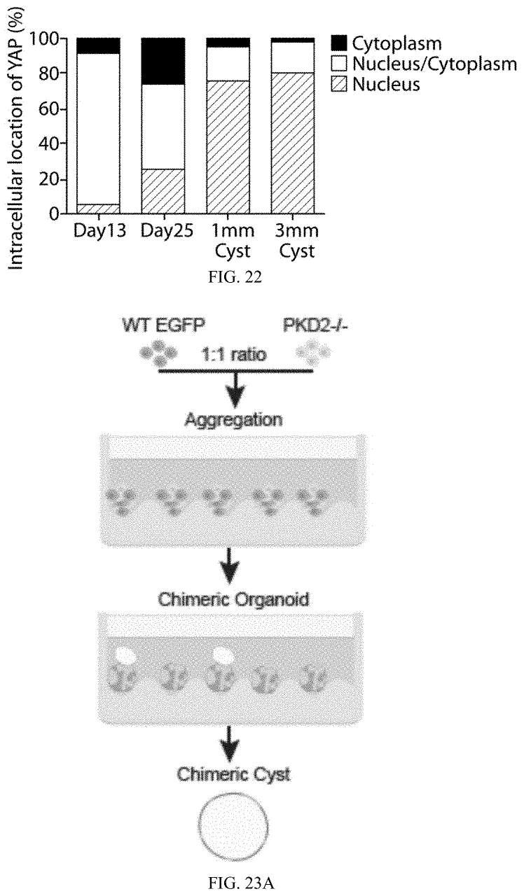

To obtain a further insight into the cyst forming process, we performed an extended culture of PKD1−/− and PKD2−/− cysts to obtain free-growing cysts detached from the main kidney organoid (FIG. 20). Cysts were propagated for extended periods (months) generating centimeter sizes cysts that could be passaged in vitro (FIG. 20). mRNA from PKD1−/− and PKD2−/− mutant organoids and independent cystic structures was isolated, sequenced and cyst-enriched genes identified through bioinformatic analysis. Each of the independent organoid cultures showed a strong correlation with each other and with wild-type kidney organoids, distinct from PKD1−/− and PKD2−/− cysts which were grouped together. Volcano plots highlighted key genes up or down regulated in cysts to organoid comparisons (FIGS. 21A and 21B). Expression of key proximal tubule regulatory factors such as Hnf4a was decreased in cysts while the injury and stress markers Havcr1 and Spp1, STAT1, a key transcriptional mediator of multiple growth factor and cytokine actions, and TNFRSF12A, a receptor linked to NF-kB pathway activation and growth of several tumor types were markedly up-regulated in cysts. Comparative analysis of PKD1−/− and PKD2−/− cyst-enriched mRNA expression profiles showed a highly significant overlap in shared gene sets (FIG. 21C). Interestingly, DAVID and INGENUITY pathway analysis predictions identified the Hippo pathway as a potential regulatory target in the cyst forming process. Hippo signaling is linked to mechano-sensation, and active Hippo signaling silences the growth and pro-survival actions of YAP/TAZ transcriptional co-activators at TEAD bound enhancers. Consistent with HIPPO silencing and YAP activation, we observed a prominent up-regulation of known YAP target genes in PKD1−/− and PKD2−/− (FIGS. 21D and 21E) and strong nuclear accumulation of YAP1 in free growing PKD1−/− and PKD2−/− cysts. The tested known YAP target genes are included in the list: SERPINE 1, ANKRD33B, TNFRSF12A, GADD45B, CCN1, CCN2, LAYN, COL12A1, ANXA3, CRIM1, RAB11FIP1, WWC1, CDCl₄2EP3, TUBB6, TMEM267, AMTL2, AJUBA, TEAD4, POLH, WWC2, HACD1, RCAN1, JADE1, ARSJ, WSB2, PKP4, ODC1, WTIP, DIAPH3, ZNHIT6, TMEM106C, MRRF, RABGEF1, SFXN4, NEDD4L, LARP4B, HAUS4, B3GALNT2, IQGAP3, HSPA9, PAWR, MTHFD1, TBC1D31, ABHD10, TMEM209, MCM3, MRPL24, PANK2, SEH1L, CHUK, ATAD2, TSEN2, DNAJA3, LSG1, CYP20A1, MRPL9, ICK, NUP93, HSPA14, DPH5, TUBB, RRP1B, MED27, CCDCl₈5C, PRMT1, TDP1, P3H2, LZIC, RALGPS2, MIIP, SMTN, NASP, CNOT9, BOD1, TMEM200B, LRIG3, C12orf45, SUV39H2, GADD45A, BASP1, BCAT1, and SOCS2.

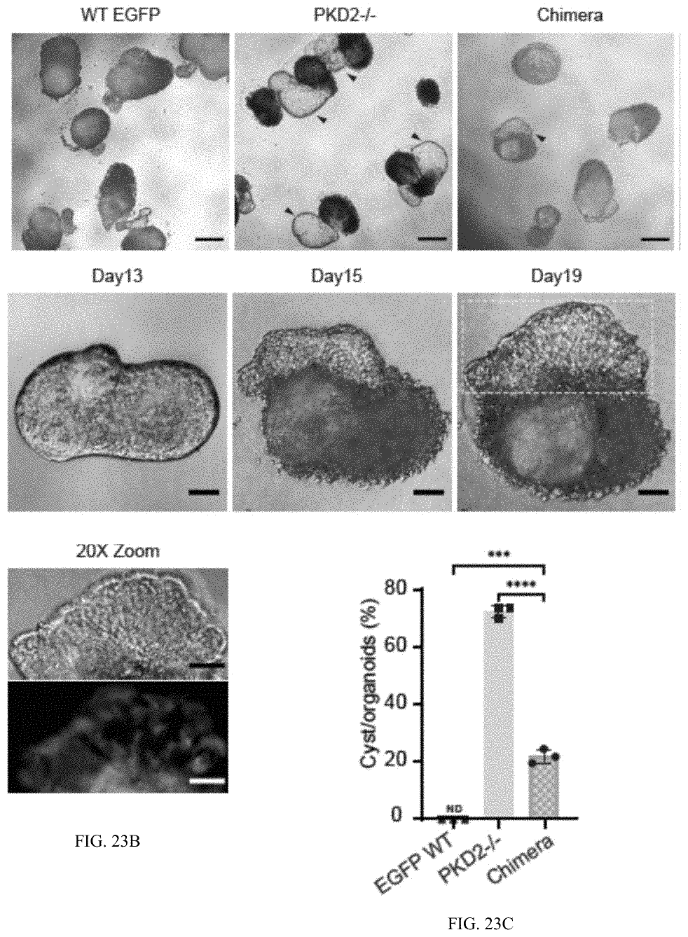

Though the Hippo pathway has emerged as a pathway of interest in cystogenesis in PKD mouse model. YAP1 activity has not been explored in the context of human PKD. To examine the relationship between YAP1 nuclear accumulation and cyst formation in organoids in vitro, we examined YAP1 at different stages of cyst outgrowth. When cysts first emerged on day 13 of organoid culture, immunohistochemistry identified YAP1 in emerging cysts, but not within the nucleus. When the size of cysts approached that of the parent organoid around day 25 of culture, cyst showed sparse mosaic patches of nuclear YAP1 activity. By the time cysts reached 1 mm in size, almost all epithelial nuclei in both PKD1−/− and PKD2−/− cysts showed nuclear accumulation of YAP1 (FIG. 22). Thus, cyst initiation may be independent of YAP1 but subsequent YAP1 nuclear accumulation may drive cystic growth providing a growth advantage to YAP1+ cells.

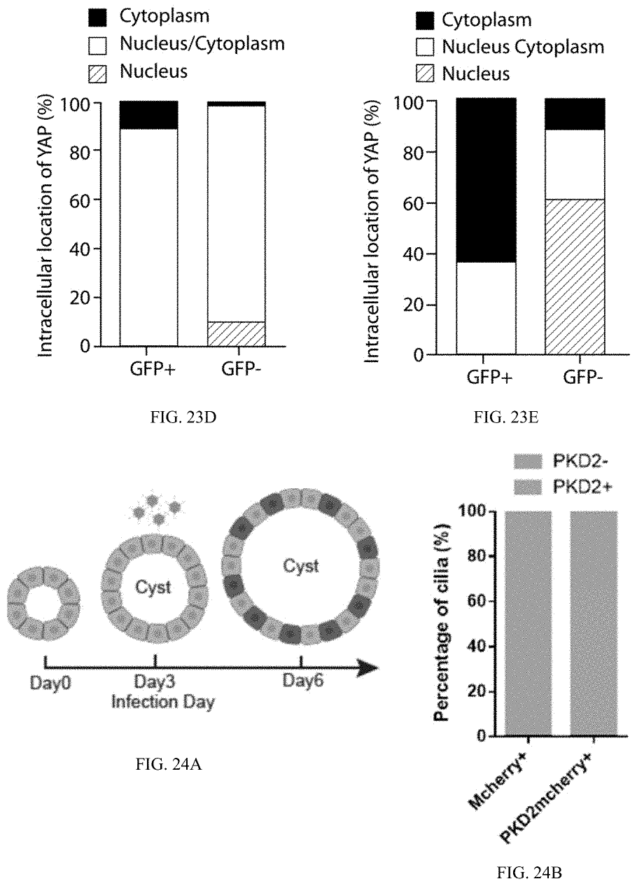

To explore the relationship between the loss of PKD gene activity and YAP1 nuclear activity, we generated chimeric kidney organoids combining PKD2−/− mutant embryo stem cells with wild type embryo stem cells (FIG. 23A). Analysis of chimeric mouse kidneys has shown that wild-type cells contribute to early cyst formation though there appears to be a selection against wildtype cells over time. Cysts formed in wild-type and PKD2−/− chimeric organoids (FIG. 23B), predominantly from LTL+ proximal tubule epithelium, though with reduced frequency, and with relatively few contributing wildtype (GFP+) cells (FIG. 23C). As expected, at 19 days of culture, most YAP1 within wildtype (GFP+) and PKD2−/− (GFP−) cysts was cytoplasmic (FIG. 23D). Interestingly, when cysts reached 1 mm in diameter, robust YAP1 nuclear localization was observed but only in PKD1−/− cells (FIG. 23E). Adjacent GFP+ wildtype cells in the LTL+ epithelium showed no enhanced nuclear accumulation of YAP1. These experiments indicate a direct, cell autonomous requirement for PKD2 to suppress YAP1 nuclear localization suggesting that YAP1 localization is not determined by more general sensing of mechanical tension within the cyst. Together these data support targeting of YAP1 nuclear activity as potential avenue to blocking cyst growth in PKD.

Restoring PKD2 Expression to Reverse Proliferation and Increased YAP1 Activity

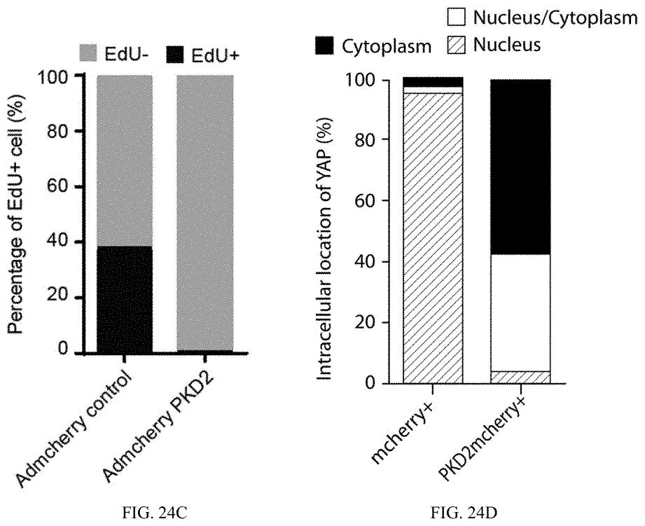

To determine the effect of restoring PKD2 activity on YAP1, we infected PKD2−/− cysts with either an adenovirus vector supporting mCherry reporter gene expression (control: Ad-mCherry) or one introducing both mCherry and PKD2 genes (experimental Ad-PKD2-mCherry) (FIG. 24A). Bright field and fluorescent images showed widespread infection throughout the whole cyst. As expected, no PKD2 protein was observed in mCherry + or − cells, while Ad-PKD2-mCherry infected cells showed both mCherry and PKD2. PKD2 localized within the ARL13B-labeled primary cilia in ˜60% of mCherry+ PKD2+ cells (FIG. 24B); the primary cilium has been reported to be the sub-cellular domain for PKD2 action consistent with the cystic phenotype observed in wide variety of mutants lacking normal primary cilium structure and/or function. PKD2 accumulation in the primary cilium is thought to regulate the co-accumulation of PKD1. Consistent with previous studies, PKD1 was only present in the primary cilium in conjunction with PKD2 infection.

Increased tubular epithelial cell proliferation is a key hallmark of ADPKD, and several studies indicate that the PKD1/PKD2 complex may regulate cell growth. Consistent with this view is the detection of elevated nuclear YAP1 levels in cystic cells of both PKD mutants. To address cell proliferation, we compared EdU incorporation as a measure of DNA replication in Ad-PKD2-mCherry or Ad-mCherry infected PKD2−/− cysts through FACS analysis of dissociated cysts. Markedly reduced EdU incorporation (Alexa-647) was observed in mCherry+ cells from Ad-PKD2-mCherry infection demonstrating that restoring PKD function can block DNA replication in cystic cells. We next sought to determine whether the presence of PKD2 altered the localization of YAP. Interestingly, we observed both a loss of YAP nuclear localization in cells mCherry+ cells following Ad-PKD2-mCherry, but not Ad-mCherry infection, as well as broader suppression of YAP1 levels in the cystic epithelium. (FIG. 24C, 24D). These results indicate that restoring PKD2 activity may have both cell autonomous and non-cell autonomous effects on the modification of YAP levels and YAP driven growth control.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method of generating a nephronic lineage organoid, comprising:
culturing a quantity of pluripotent stem cells (PSCs) in the presence of a first small molecule, and culturing the quantity of PSCs in the presence of at least one growth factor, to generate nephron progenitor-like cells;
culturing the nephron progenitor-like cells in the presence of the at least one growth factor and a second small molecule, and generating a pellet of cells by placing the nephron progenitor-like cells into a microwell;
incubating the pellet of cells in the presence of the at least one growth factor; and
further culturing the pellet of cells in the absence of the at least one growth factor to generate a nephronic lineage organoid.

2. The method of claim 1, wherein the at least one first growth factor comprises activin, fibroblast growth factor (FGF) 9, or both.

3. The method of claim 1, wherein at least one of the first small molecule and the second small molecule is CHIR99021.

4. The method of claim 1, wherein the culturing of the nephron progenitor-like cells in the presence of the at least one growth factor and a second small molecule comprises addition of FGF9 and CHIR99021 to the nephron progenitor-like cells.

5. The method of claim 1, wherein the PSCs comprises human embryonic stem cells (hESCs), and optionally at least some of the hESCs have a mutant polycystin-1 gene and/or a mutant polycystin-2 gene.

6. The method of claim 1, wherein the PSCs comprises a first quantity of PSCs with a mutant polycystin-1 gene or a mutant polycystin-2 gene, and a second quantity of PSCs that have a normal polycystin-1 gene and a normal polycystn-2 gene and that optionally express a marker.

7. The method of claim 6, wherein the PSCs express one or more markers selected from the group consisting of a podocyte marker, a proximal tube marker, a loop of Henle marker, and a distal tubule marker, and wherein
the podocyte marker comprises V-maf musculoaponeurotic fibrosarcoma oncogene homolog B (MafB) or Wilm's tumor suppressor gene (WT1),
the proximal tube marker comprises cubilin (CUBN),
the loop of Henle marker comprises SLC12A1, and
the distal tubule marker comprises SLC12A3.

8. The method of claim 1, wherein the PSCs express one or more of transcription factor MafB; cubilin (CUBN); solute carrier family 12 member 1 (SLC12A1); and LAMB1.

9. The method of claim 1, wherein the method generates the nephronic lineage organoid within 20 days from the initiation of culturing of the quantity of the PSCs.

10. The method of claim 1, wherein the method further comprises culturing the nephronic lineage organoids in a medium containing a cellulose-based thickener and/or the step of further culturing the pellet of cells includes culturing in a medium containing the cellulose-based thickener, coupled with imaging the nephronic lineage organoids or the pellet under a microscope or camera over an extended period of time.

11. An organoid, comprising:
a quantity of nephrons, nephron progenitor cells, or both,
a quantity of interstitial cells,
a quantity of endothelial cells,
a quantity of neuron-like cells that express NEUROD1 and NEUROG1,
a quantity of neural crest-like cells that are positive for SOX10,
a quantity of muscle-like cells that are positive for TNNI1 and ACTC1,
and optionally a trace quantity of human pluripotent stem cells,
wherein the organoid is in a three-dimensional form and is positive for one or more markers selected from the group consisting of WT1, PAX2, PAX8, MAFB, CUBN, HNF4A, GATA3, SLC3A1, SLC12A1, SLC12A3, PDGFRA, SOX17, and CDH5.

12. The organoid of claim 11, wherein the organoid has a tubule or renal vesicle-like structure that is CDH1+, or the organoid comprises a quantity of cells that have mutant polycystin 1 gene and/or mutant polycystin 2 gene.

13. The quantity of organoids of claim 11, wherein at least 10%, 20%, 30%, 40%, 50%, or 60% of the organoids or comprise a tubule and/or cyst.

14. A method of screening for a candidate drug for treating, reducing the incidence or severity of polycystic kidney disease, the method comprising:
contacting a molecule of interest with a nephronic lineage organoid;
measuring a level of a biomarker transcribed or expressed in the nephronic lineage organoid and/or evaluating cyst formation or progression before the contact with the molecule of interest;
measuring a level of the biomarker transcribed or expressed in the nephronic lineage organoid and/or evaluating the cyst formation or progression in the presence of the molecule of interest,
wherein the nephronic lineage organoid is generated by a process of:
culturing a quantity of pluripotent stem cells (PSCs) in the presence of a first small molecule, and culturing the quantity of PSCs in the presence of at least one growth factor, to generate nephron progenitor-like cells;
culturing the nephron progenitor-like cells in the presence of the at least one growth factor and a second small molecule, prior to or after generating a pellet of cells by placing the nephron progenitor-like cells into a microwell;
incubating the pellet of cells in the presence of the at least one growth factor; and
further culturing the pellet of cells in the absence of the at least one growth factor, whereby a nephronic lineage organoid is generated.

15. The method of claim 14, wherein the biomarker comprises a yes-associated protein 1 (YAP1), a signal transducer and activator of transcription 1 (STAT1), polycystin1 (PKD1), polycystin2 (PKD2), hepatocyte nuclear factor 4 alpha (Hnf4a), hepatitis A virus cellular receptor 1 (Havcr1), secreted phosphoprotein 1 (SPP1), tumor necrosis factor receptor superfamily member 12A (TNFRSF12A), or a combination thereof.

16. The method of claim 15, wherein at least some of the quantity of PSCs to generate the nephronic lineage organoid comprises mutant polycystic 1 gene and/or mutant polycystic 2 gene, and wherein the molecule of interest is identified as a candidate drug for treating, reducing the incidence or severity of polycystic kidney disease when:
a) mRNA and/or protein level of HNF4A is increased in the presence of the molecule of interest, compared to that before the contact with the molecule of interest, or
b) mRNA and/or protein level of HAVCR1, SPP1, STAT1, TNFRSF12A, and/or YAP1 is decreased in the presence of the molecule of interest, compared to that before the contact with the molecule of interest;
and wherein the molecule of interest is identified as not a candidate drug for treating, reducing the incidence or severity of polycystic kidney disease when:
c) mRNA and/or protein level of HNF4A is not increased in the presence of the molecule of interest, compared to that before the contact with the molecule of interest, or
d) mRNA and/or protein level of HAVCR1, SPP1, STAT1, TNFRSF12A, and/or YAP1 is not decreased in the presence of the molecule of interest, compared to that before the contact with the molecule of interest.

17. The method of claim 14, wherein the molecule of interest comprises a protein kinase inhibitor.

18. The method of claim 14, wherein at least some of the quantity of PSCs to generate the nephronic lineage organoid comprises mutant polycystic 1 gene and/or mutant polycystic 2 gene, and the molecule of interest comprises an adenovirus-based vector, a lentivirus-based vector, a retrovirus-based vector, an adeno-associated virus, a pox virus-based vector, an alphavirus-based vector, or a herpes virus-based vector, and
wherein the molecule of interest is identified as a candidate drug for treating, reducing the likelihood or severity of polycystic kidney disease when the molecule of interest decreases expression level of a YAP1 target gene, said YAP1 target gene comprises CTGF or CYR61,
and the molecule of interest is identified as not a candidate drug for treating, reducing the likelihood or severity of polycystic kidney disease when the molecule of interest does not decrease the expression level of the YAP1 target gene.

19. A method of treating, reducing the likelihood or severity of autosomal dominant polycystic kidney disease (ADPKD) in a subject in need thereof, the method comprising:
screening for an agent for treating, reducing the incidence or severity of polycystic kidney disease according to the method of claim 14, wherein a nephronic lineage organoid is optionally generated from a quantity of PSCs obtained from the subject; and
administering to the subject an effective amount of the agent, said agent is selected from the group consisting of fascaplysin, an inhibitor of mitogen-activated protein kinase-interatcting serine/threonine-protein kinase 1 (MNK1), PD98059, RO-3306, a dual inhibitor of Cdc7/Cdk9, 4-cyano-3-methylisoquinoline, IKK-2 inhibitor VI ((5-phenyl-2-ureido)thiophene-3-carboxamide), IKK inhibitor VII, UCN-01 (7-Hydroxystaurosporine), UCN-02 (7-epi-hydroxystaurosporine), celastrol, staurosporine, and carfilzomib,
thereby reducing, slowing or inhibiting the formation or progression of cyst in one or more organs of the subject.

20. A system, comprising:
human pluripotent stem cells (PSCs) of claim 11,
a culture medium comprising methylcellulose,
a growth factor comprising fibroblast growth factor (FGF) 9,
another growth factor comprising avidin,
a small molecule comprising CHIR99021,
a micro-well plate, and
instructional material.
